(19) United States
(12) Patent Application Publication
(10) Pub. No.: US 2023/0038695 A1
(43) Pub. Date: Feb. 9, 2023

(54) VIRTUAL REALITY ACTIVITIES FOR VARIOUS IMPAIRMENTS

(71) Applicant: PENUMBRA, INC., Alameda, CA (US)

(72) Inventors: William Ka-Pui Yee, Alameda, CA (US); Georgia Mitsi, Wellesley, MA (US); Steven Chen, Alameda, CA (US); Andrew Mathis, San Jose, CA (US)

(21) Appl. No.: 17/394,558

(22) Filed: Aug. 5, 2021

(51) Int. Cl. A61M 21/02; G16H 20/70; G16H 10/60; G06F 3/01; G06F 3/0484

(52) U.S. Cl. CPC A61M 21/02; G16H 20/70; G16H 10/60; G06F 3/011; G06F 3/0484; A61M 2021/0027

(57) ABSTRACT

Systems and methods are provided for identifying a therapeutic VR activity or exercise for a subject/patient based on the subject's impairments, dynamically adjusting a VR activity for a patient, and identifying potential impairments based on a patient's performance in a VR activity. Patients may each have various physical, neurological, cognitive, and/or sensory impairments to be treated. Not all therapeutic activities may be appropriate for some patients and their impairments. A VR therapeutic activity platform may increase patient engagement and challenge patients at more appropriate times by better matching activities corresponding to a patient's impairments and dynamically adjusting each VR activity based on performance to offer a challenging and rewarding therapeutic experience.

### BACKGROUND OF THE DISCLOSURE

The present disclosure relates generally to virtual reality (VR) systems and more particularly to providing therapeutic VR activities to engage a patient experiencing at least one of various physiological and/or neurocognitive impairments.

### SUMMARY OF THE DISCLOSURE

Virtual reality systems may be used in various applications, including therapeutic activities and exercises, to assist patients with their rehabilitation and recovery from illness or injury. VR may be used to monitor and help patients retrain their brains and muscles to perform certain tasks that may be difficult in a safe, observable environment. Therapy may not always be easy or engaging for a patient, but VR activities have shown promise as engaging therapy for patients suffering from a multitude of conditions. Patients may each have various physical, neurological, cognitive, and/or sensory impairments to be treated. Even with VR, not all therapeutic activities may be appropriate for some patients and their impairments. Therapy may be too easy one day and too challenging the next. Therapeutic tasks may be calm one moment and highly stress-inducing at another point. VR therapy may provide engaging worlds, exercises and various tailored activities, but it is not immune to patient fatigue and frustration. A VR therapeutic activity platform can increase patient engagement and challenge patients at more appropriate times by better matching activities corresponding to a patient's impairments and dynamically adjusting each VR activity based on performance to offer a challenging and rewarding therapeutic experience. With a VR platform identifying and suggesting therapy activity, a therapist may be able to better focus on the patient. A VR platform may also allow a patient to independently practice portions of a guided VR activity regimen outside of a therapist's office, e.g., at home under the supervision of a family member and/or a remote supervisor.

Generally, VR systems can be used to instruct users in their movements while therapeutic VR can recreate practical exercises that may further rehabilitative goals such as physical development and neurorehabilitation. For instance, patients with physical and neurocognitive disorders may use therapy for treatment to improve, e.g., range of motion, balance, coordination, mobility, flexibility, posture, endurance, and strength. Physical therapy may also help with pain management. Some therapy, e.g., occupational therapy, may help patients with various impairments develop physically and mentally to better perform everyday living functions, and activities of daily life (ADLs). VR systems can encourage patients by depicting avatars performing tasks that a patient with various impairments may not be able to fully execute.

VR therapy can be used to treat various disorders, including physical disorders causing difficulty or discomfort with reach, grasp, positioning, orienting, range of motion (ROM), conditioning, coordination, control, endurance, accuracy, and others. VR therapy can be used to treat neurological disorders disrupting psycho-motor skills, visual-spatial manipulation, control of voluntary movement, motor coordination, coordination of extremities, dynamic sitting balance, eye-hand coordination, visual-perceptual skills, and others. VR therapy can be used to treat cognitive disorders causing difficulty or discomfort with cognitive functions such as instrumental activities of daily living (IADLs), executive functioning, short-term and working memory, sequencing, procedural memory, stimuli tolerance and endurance, sustained attention, attention span, and others. In some cases, VR therapy may be used to treat sensory impairments with, e.g., sight, hearing, smell, touch, taste, and/or spatial awareness.

A VR system may use an avatar of the patient and animate the avatar in the virtual world. Using sensors in VR implementations of therapy allows for real-world data collection as the sensors can capture movements of body parts such as hands and arms for the system to convert and animate an avatar in a virtual environment. Such an approach may approximate the real-world movements of a patient to a high degree of accuracy in virtual-world movements. Data from the many sensors may be able to produce statistical feedback for viewing and analysis by doctors and therapists. Generally, avatar animations in a virtual world may closely mimic the real-world movements, but virtual movements may be exaggerated and modified in order to aid in therapeutic activities. Visualization of patient movements through avatar animation could stimulate and promote physical and neurological repairs, recovery, and regeneration for a patient. For example, a VR activity may depict an avatar feeding a bird some birdseed from the avatar's hand based on a patient's actual movements of grabbing and shaking a seed dispenser into his corresponding open virtual palm. A VR activity may ask a patient to stack virtual ingredients for a specific sandwich by requiring the patient to reach towards bread, meats, cheeses, lettuce, and condiments in a step-by-step fashion.

VR activities have shown promise as engaging therapy for patients suffering from a multitude of conditions, bringing engaging features to a mentally and physically tough process. Therapy can be stress-inducing and still can fall victim to patient fatigue and frustration. More VR activities are being developed to address specialized impairments with tailored exercises.

With a variety of VR activities, comes a variety of exercises for therapy patients. However, not every exercise or activity is correct or properly suited for every patient. Patients may each have various physical, neurological, cognitive, and/or sensory impairments to be treated. Even with VR, not all therapeutic activities may be appropriate for some patients and their impairments. For instance, a patient with back issues may not be a good fit for an activity exercising trunk control or cross-body reach. A patient with impaired range of motion in her shoulder, may not need exercises or activities designed to focus on visual scanning or object recognition. An activity that works on voluntary movements may not be appropriate for a patient with impaired working memory and sequencing conditions.

To help present and identify a patient's conditions and impairments to a therapist or supervisor of a VR system, a VR system may incorporate additional data such as a patient's diagnoses and health data. Some VR systems may use, for example, a patient profile to store a patient's diagnosed impairments, therapy records, movement data, and activity performance data. Activities within VR applications may each have data stored to describe the goals and treatment in each activity or task. Prior to a therapist or supervisor initiating a therapy session, she should review patient impairments and impairments treated by the activity to ensure a good fit and avoid potentially injurious conflicts. However, as more patients are added and more VR activities become available, there exists a need for a VR platform to match patients with activities based on the impairments.

As disclosed herein, a VR therapeutic activity platform can increase patient engagement and challenge patients at more appropriate times by better matching activities corresponding to a patient's impairments to offer a challenging and rewarding therapeutic experience. Generally, a VR platform will compare impairments of a patient's profile to each activity's list of impairments to be treated, determine if the impairments match, e.g., above a threshold, and provide a subset of suggested activities matching the patient's impairments. With a VR platform identifying and suggesting therapy activities based on the patient's impairments, a therapist may be able to better focus on the patient. A VR platform may also allow a patient to independently practice portions of a guided VR activity regimen outside of a therapist's office, e.g., at home under the supervision of a family member and/or a remote supervisor.

In some embodiments, impairments from each activity's list and impairments identified in the patient profile may be compared. In some embodiments, matches are identified and counted. A patient profile indicating impairments diminishing the patient's range of motion, trunk control, functional reach, executive functioning, sequencing, and working memory may be presented with one VR activity treating, e.g., range of motion, voluntary movement, coordination, and balance or another activity treating, e.g., trunk control, functional reach, cross-body motion, and shoulder flexion and extension. In some embodiments, the suggested therapy activities may be ranked or presented in an order to be played. In some embodiments, matches from each activity's list may be prioritized or weighted based on prevalence within the activity or in the patient profile. For instance, matches are identified and weighted based on a tier of each impairment (e.g., prioritization).

Even when an appropriate VR activity is presented to a patient, there can be challenges. Therapy may be too easy in one activity and too challenging in the next. Therapeutic tasks may be calm one moment and highly stress-inducing at another point. When a patient is struggling, engagement may plummet and risks of the patient not completing therapy may rise. Metrics in activity performance data may be used to monitor when a patient is struggling or coasting. Performance data may incorporate measurements such as scores, hit rates, body movement data, range of motion, success rates, times, speed, reaction times, and other data. Generally, performance data is based on sensor data received from a plurality of VR sensors placed on the patient's body. Performance data may be exhibited as a score or kept secretly from the patient (e.g., only viewable by the system and/or therapist). Activity performance data may comprise additional biometric feedback. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, facial reflexive movement tracking, facial expression monitoring, respiratory monitors, light sensors, cameras, sensors, and other biometric devices. Biometric feedback, along with other performance data, can indicate more subtle changes to the patient's body, physiology, or mental state, e.g., when a patient is stressed, comfortable, distracted, tired, over-worked, under-worked, over-stimulated, confused, overwhelmed, excited, engaged, disengaged, and more. Performance data, including scores, records, biometric may be stored with profile and/or application data in a secure database. Using performance data, a system can determine when a patient is comfortable in therapy and when he might be too uncomfortable to engage in the therapy. There exists a need to ensure appropriate levels of VR activity success to promote continued VR therapy participation and development.

One approach may be to make the activities super easy, however, that may minimize therapeutic impact of the exercise. For instance, if tracking a moving object (e.g., a squirrel) with your head or eyes in a VR activity is too easy, skills in sustaining attention and exercise of cervical range of motion may not be accomplished. Likewise, a too-easy exercise of Hide and Seek may not engage the patient and encourage further activities. There exists a need to, e.g., reduce the challenges in an activity when needed and to increase the difficulty when therapy is too easy.

As disclosed herein, a VR therapeutic activity platform can dynamically adjust a VR activity for a patient. Generally, a VR platform can determine if activity performance data falls outside the VR activity's optimal performance range and dynamically adjust the VR activity to encourage patient engagement. Some embodiments may adjust activity challenge level based on the patient's profile, e.g., a patient's impairments. Some embodiments may use rules to adjust the activity experience when performance is too poor or too good. For instance, in some activities, if the patient has a higher percentage of touching objects with one hand versus the other, more objects for the weak hand may be generated. Speed or frequency of object generation may be adjusted with, e.g., more objects if performance is above the optimal range and fewer objects if performance is suboptimal. Frequency may be adjusted if metrics (e.g., heart rate, blood pressure, respiration, perspiration, eye movements, facial movements, facial expressions, etc.) indicate elevated stress. With regard to the orientation of the ice creams, if performance metrics indicate that the wrist rotation is not matching well (e.g., less the 50% matches within 10% of the angle), a dynamic adjustment rule may indicate the objects should be rotated less. In some embodiments, assistance for rotation may occur to help ease the matching or better demonstrate the goal of the exercise. Size of the objects may be adjusted if performance metrics identify that a patient may not be, e.g., seeing the object.

Some embodiments may provide additional guidance with activity cues, shapes, focus lights, and cursors when performance is diminished. For instance, an object may flash (more) at a time when the patient is supposed to touch it. In some cases, rules may dictate that environmental distractions such as extra animations and sounds may be limited if performance is suboptimal. For instance, background character and environment animations such as bunnies, peppermint sticks, gingerbread men, etc., may not dance as much (or even appear) if the performance metrics, e.g., for eye tracking indicates performance is hindered due to too many distractions. In some embodiments, colors may change to promote more positive feelings and inspire confidence. For instance, a rule may decide that a flower object should be changed from red to yellow when metrics identify a patient may be feeling stressed, as yellow may be considered a more calming color. Rules for dynamic adjustments may be stored with application data in a data structure in a database.

While performance within VR activity being monitored there also exists a need to track potential impairments that may be indicated by poor performance of certain exercises. There exists a need to record new potential impairments that the VR platform may identify and determine as potentially problematic for the patient, e.g., warning the therapist and doctors.

As disclosed herein, a VR therapeutic activity platform can identify potential impairments based on a patient's performance in a VR activity. Generally, identifying a potential impairment comprises determining if particular performance data falls below a threshold for accuracy, speed, and/or comprehension in a VR activity and supplementing a patient's impairment profile, e.g., as a potential impairment, if a threshold is not met. For instance, in one object-touching activity, if accuracy of objects touched is below a threshold of 35%, there may be issues with functional reach, coordination, and/or control. In a virtual xylophone activity, if the duration between correct notes is above 45 seconds, there may be issues with, e.g., working memory and/or sequencing. In an activity where the patient chooses and feeds a berry to animals, a count of incorrect berries greater than, e.g., 7, may indicate issues with regard to object recognition, color and shape matching, and/or sustained attention skills. The VR platform may supplement a patient's impairment profile, e.g., as a potential impairment, with one or more of those conditions for urgent follow-up with a doctor.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1A is an illustrative depiction of a user interface for a VR therapy platform, in accordance with some embodiments of the disclosure;

FIG. 1B is an illustrative depiction of a user interface for a VR therapy platform, in accordance with some embodiments of the disclosure;

FIG. 2 depicts an illustrative data structure for a patient profile, in accordance with some embodiments of the disclosure;

FIG. 3 depicts an illustrative data structure for VR applications and activities, in accordance with some embodiments of the disclosure;

FIG. 4 depicts an illustrative flowchart of a process for selecting an appropriate VR activity for a patient, in accordance with some embodiments of the disclosure;

FIG. 5 depicts an illustrative flowchart of a process for dynamically adjusting a VR activity for a patient, in accordance with some embodiments of the disclosure;

FIG. 6 depicts an illustrative flowchart of a process for identifying potential impairments based on patient performance in a VR activity, in accordance with some embodiments of the disclosure;

FIG. 7A is a diagram of an illustrative system, in accordance with some embodiments of the disclosure;

FIG. 7B is a diagram of an illustrative system, in accordance with some embodiments of the disclosure;

FIG. 7C is a diagram of an illustrative system, in accordance with some embodiments of the disclosure;

FIG. 7D is a diagram of an illustrative system, in accordance with some embodiments of the disclosure;

FIG. 8A is a diagram of an illustrative system, in accordance with some embodiments of the disclosure;

FIG. 8B is a diagram of an illustrative system, in accordance with some embodiments of the disclosure;

FIG. 8C is a diagram of an illustrative system, in accordance with some embodiments of the disclosure;

FIG. 9 is a diagram of an illustrative system, accordance with some embodiments of the disclosure; and

FIG. 10 is a diagram of an illustrative system, in accordance with some embodiments of the disclosure;

FIG. 11A depicts illustrative user interfaces for a VR therapy activity, Music in Motion, in accordance with some embodiments of the disclosure;

FIG. 11B depicts illustrative user interfaces for a VR therapy activity, Music in Motion, in accordance with some embodiments of the disclosure;

FIG. 12A depicts illustrative user interfaces for a VR therapy activity, Pleasant Cove, in accordance with some embodiments of the disclosure;

FIG. 12B depicts illustrative user interfaces for a VR therapy activity, Pleasant Cove, in accordance with some embodiments of the disclosure;

FIG. 13A depicts illustrative user interfaces for a VR therapy activity, Mindful Market, in accordance with some embodiments of the disclosure;

FIG. 13B depicts illustrative user interfaces for a VR therapy activity, Mindful Market, in accordance with some embodiments of the disclosure;

FIG. 14 depicts illustrative user interfaces for a VR therapy activity, Pinball, in accordance with some embodiments of the disclosure;

FIG. 15A depicts illustrative user interfaces for a VR therapy activity, Island Antics, in accordance with some embodiments of the disclosure;

FIG. 15B depicts illustrative user interfaces for a VR therapy activity, Island Antics, in accordance with some embodiments of the disclosure;

FIG. 16A depicts illustrative user interfaces for a VR therapy activity, Serene Lake, in accordance with some embodiments of the disclosure;

FIG. 16B depicts illustrative user interfaces for a VR therapy activity, Serene Lake, in accordance with some embodiments of the disclosure;

FIG. 17 depicts illustrative user interfaces for a VR therapy activity, Mimic, in accordance with some embodiments of the disclosure;

FIG. 18 depicts illustrative user interfaces for a VR therapy activity, Float, in accordance with some embodiments of the disclosure;

FIG. 19 depicts illustrative user interfaces for a VR therapy activity, Flourish, in accordance with some embodiments of the disclosure; and

FIG. 20 depicts illustrative user interfaces for a VR therapy activity, Mending Garden, in accordance with some embodiments of the disclosure.

### DETAILED DESCRIPTION

Various systems and methods disclosed herein are described in the context of a therapeutic system for helping patients, but this application is only illustrative. In context of the VR system, the word “therapy” may be considered equivalent to physical therapy, cognitive therapy, neurological therapy, sensory therapy, behavioral therapy, occupational therapy, preventative therapy, assessment for therapies, and/or any other methods to help manage an impairment or condition, as well as a combination of one or more therapeutic programs. Such a VR system may be suitable with, for example, therapy, coaching, training, teaching, and other activities. Such systems and methods disclosed herein may apply to various VR applications.

In context of the VR system, the word “patient” may be considered equivalent to a subject, user, participant, student, etc. and the term “therapist” may be considered equivalent to doctor, physical therapist, clinician, coach, teacher, supervisor, or any non-participating operator of the system. A therapist may configure and/or monitor via a clinician tablet, which may be considered equivalent to a personal computer, laptop, mobile device, gaming system, or display. Some disclosed embodiments include a digital hardware and software medical device that uses VR for health care, focusing on physical and neurological rehabilitation. The VR device may be used in a clinical environment under the supervision of a medical professional trained in rehabilitation therapy. In some embodiments, the VR device may be configured for personal use at home, e.g., with remote monitoring. A therapist or supervisor, if needed, may monitor the experience in the same room or remotely. In some cases, a therapist may be physically remote or in the same room as the patient. For instance, some embodiments may need only a remote therapist. Some embodiments may require a remote therapist with someone, e.g., a nurse or family member, assisting the patient to place or mount the sensors and headset and/or observe for safety. Generally, the systems are portable and may be readily stored and carried by, e.g., a therapist visiting a patient.

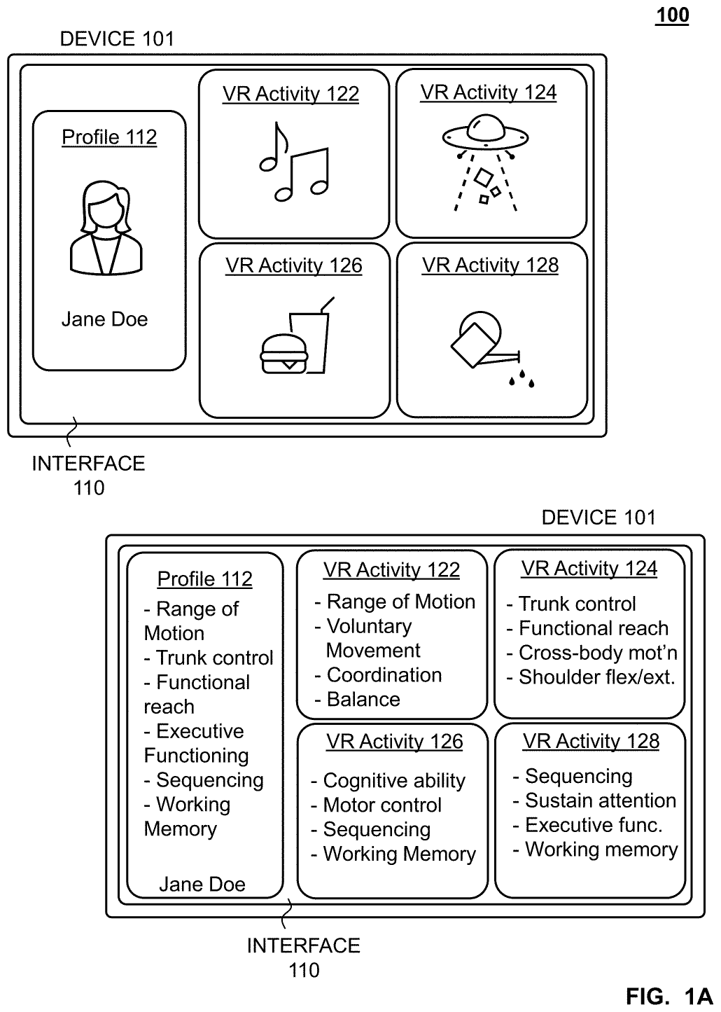

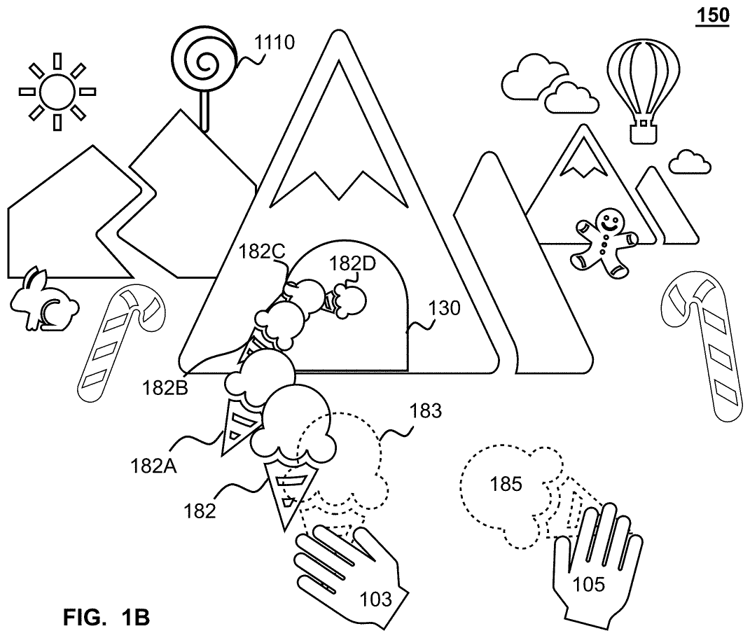

FIG. 1A is an illustrative depiction of a user interface for a VR therapy platform, in accordance with some embodiments of the disclosure. By way of a non-limiting example, Scenario 100 of FIG. 1A illustrates a user interface of a virtual reality application as depicted to a patient view in the head-mounted display (HMD), e.g., “Patient View.” Scenario 100 may also be considered a user interface of the same VR application as depicted to a spectator, such as a therapist. For instance, a spectator, such as a therapist, may view Scenario 100 and see a reproduction or mirror of a patient's view in the HMD, e.g., “Spectator View.” Spectator View may replicate a portion of the display presented to the patient, “Patient View,” that fits on a display, e.g., a supervisor tablet. Scenario 100 may be referred to as “Patient View” or “Spectator View.”

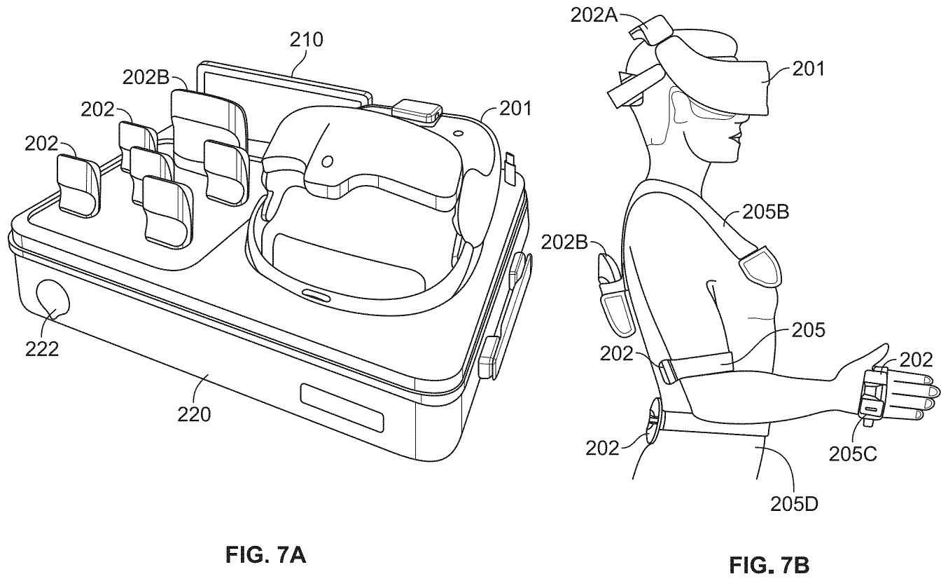

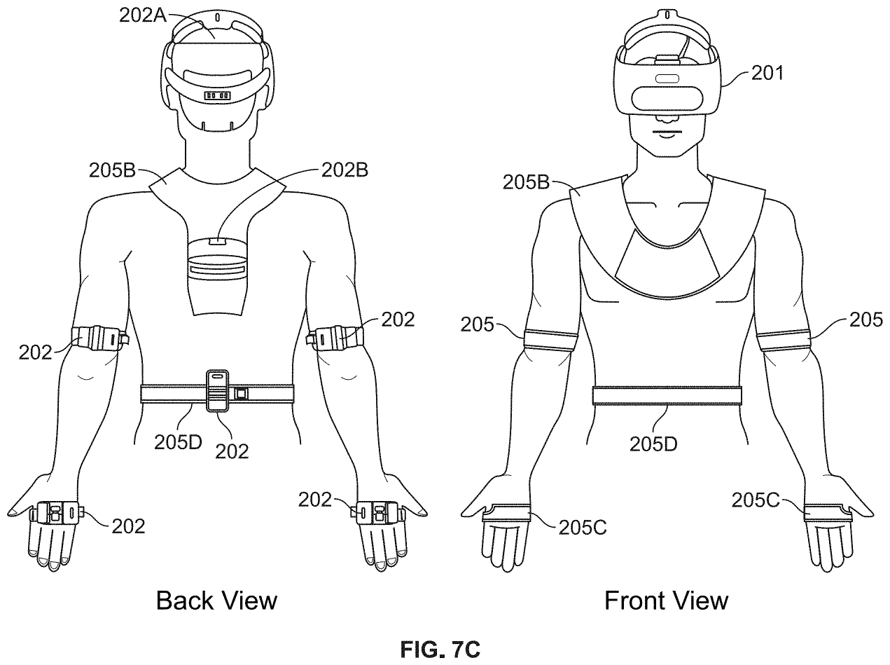

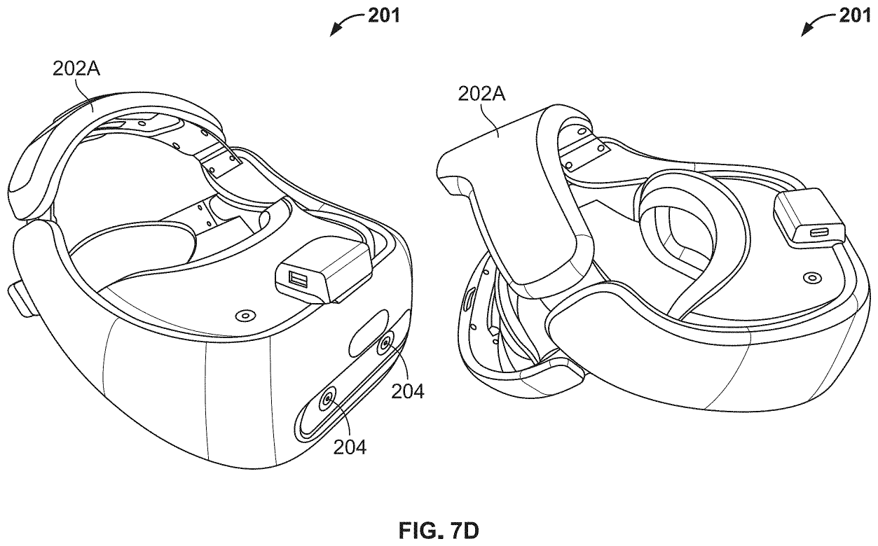

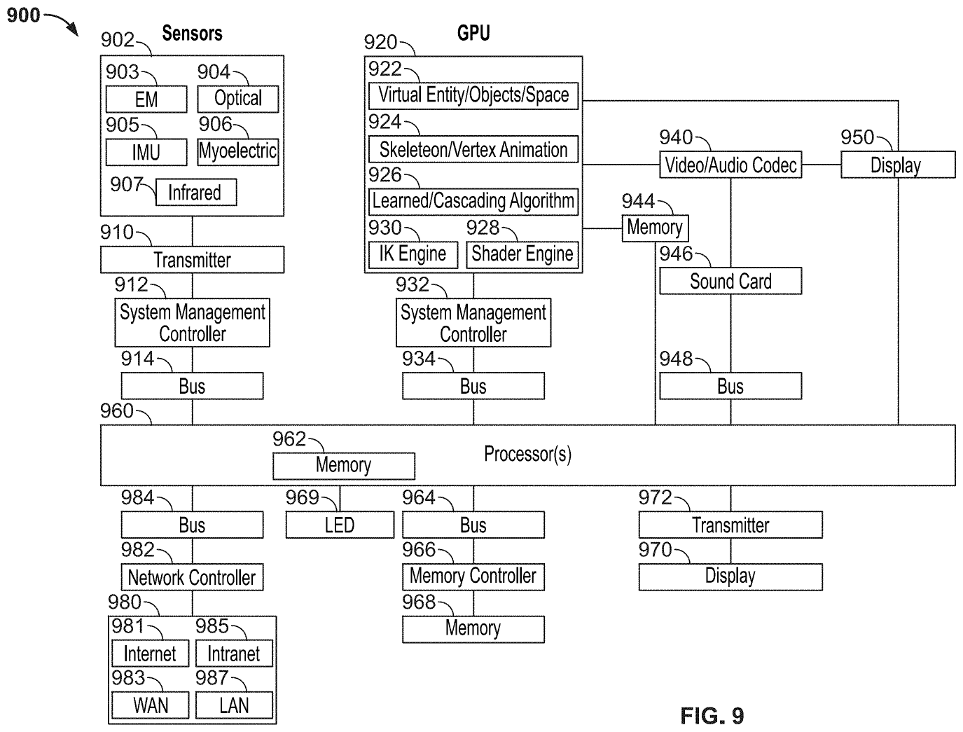

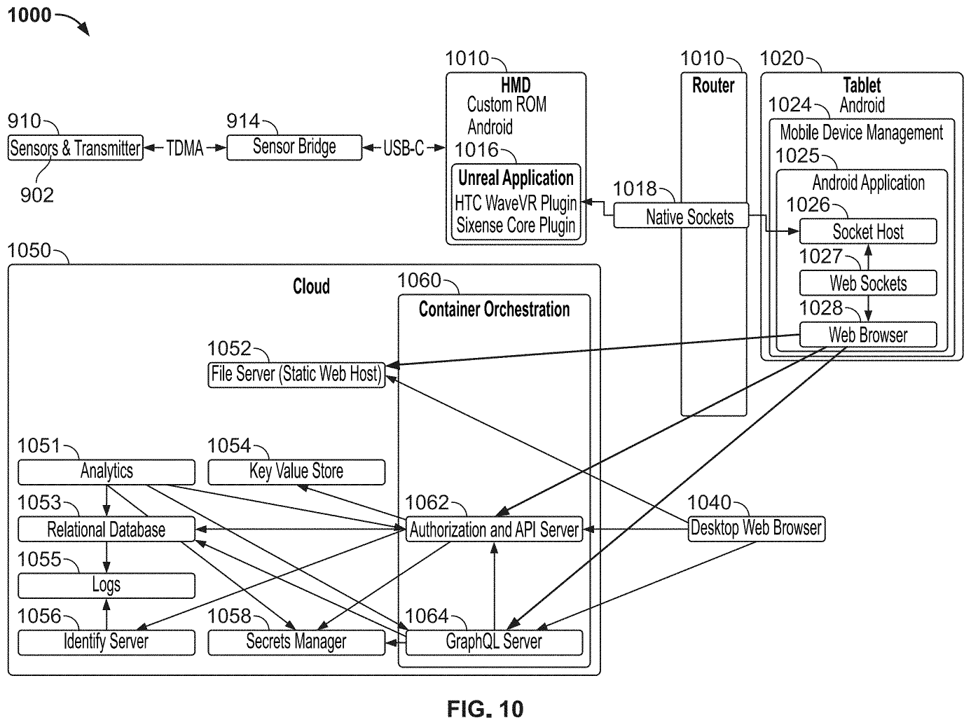

Patient View is the view of the VR world from the VR headset. A VR environment rendering engine (sometimes referred to herein as a “VR application”) on device 101, e.g., an HMD, such as the Unreal® Engine, may use the position and orientation data to generate a virtual world including an avatar that mimics the patient's movement and view. Unreal Engine is a software-development environment with a suite of developer tools designed for developers to build real-time 3D video games and applications, virtual and augmented reality graphics, immersive technology simulations, 3D videos, digital interface platforms, and other computer-generated graphics and worlds. A VR application may incorporate the Unreal Engine or another three-dimensional environment developing platform, e.g., sometimes referred to as a VR engine or a video game engine. Some embodiments may utilize a VR application, stored and executed by one or more of the processors and memory of a headset, server, tablet and/or other device to render Scenario 100. For instance, a VR engine may be incorporated in one or more of head-mounted display 201 and clinician tablet 210 of FIGS. 7A-D and/or the systems of FIGS. 9-10. A VR engine may run on a component of a tablet, HMD, server, display, television, set-top box, computer, smartphone, or other device. A VR engine may also generate interface 110 of scenario 100.

Spectator View, as seen, e.g., in scenario 100, may be a copy of what the patient sees on the HMD while participating in a VR activity, e.g., Patient View. In some embodiments, Scenario 100 may be depicted on a therapist's tablet or display, such as clinician tablet 210 as depicted in FIG. 7A. For instance, scenario 100 may be a reproduction of Patient View from a participant's HMD, such as headset 201 of FIGS. 7A-D. In some embodiments, an HMD may generate a Patient View as a stereoscopic three-dimensional (3D) image representing a first-person view of the virtual world with which the patient may interact. An HMD may transmit Patient View, or a non-stereoscopic version, as Spectator View to the clinician tablet for display. Spectator View may be derived from a single display, or a composite of both displays, from the stereoscopic Patient View.

Interface 110 of scenario 100 of FIG. 1 may be considered a menu for a VR therapy platform. In some embodiments, interface 110 depicts suggested VR activities 122, 124, 126, and 128 based on an identified patient profile 112. Each of profile 112 and VR activities 122, 124, 126, and 128 based on patient profile 112 may be presented with a representative image or icon. Each of profile 112 and VR activities 122, 124, 126, and 128 based on patient profile 112 may be presented with descriptions, e.g., impairments to be treated.

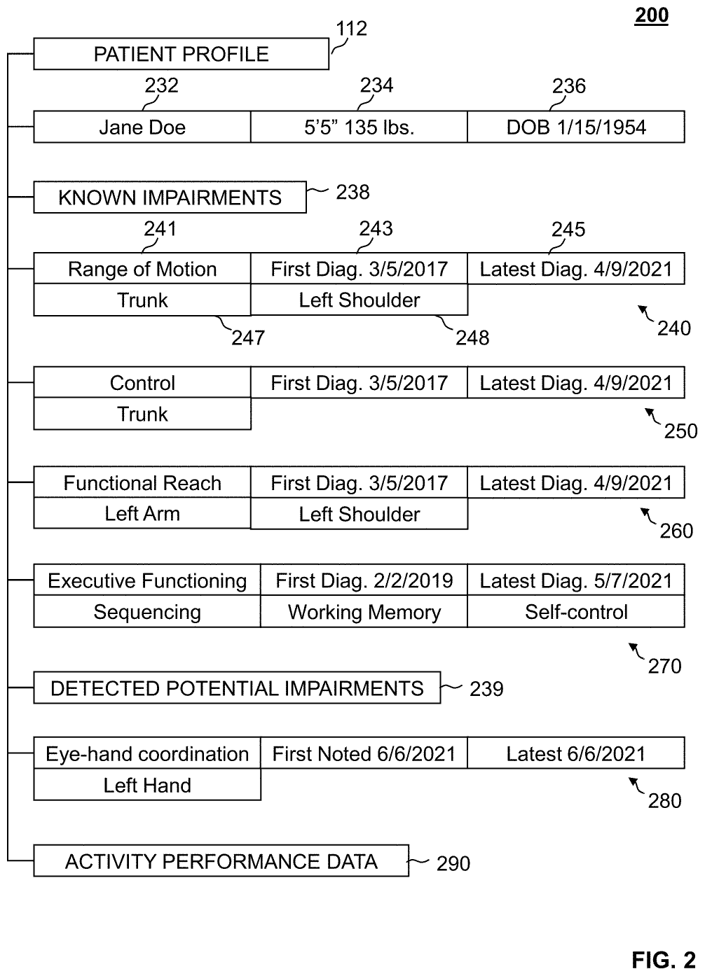

Interface 110 depicts patient profile 112 for “Jane Doe.” Patient profile 112 is shown to be documented with the patient experiencing, e.g., impairments diminishing the patient's range of motion, trunk control, functional reach, executive functioning, sequencing, and working memory. Patient profile 112 may include further impairment data, health data, VR activity data, and other relevant data. An exemplary data structure for storing patient profile 112 is depicted in scenario 200 of FIG. 2. Patient profile 112 may be accessed and loaded, e.g., as a patient logs in to interface 110, e.g., the VR therapy platform. In some embodiments, loading patient profile 112 may be initiated by a therapist or supervisor.

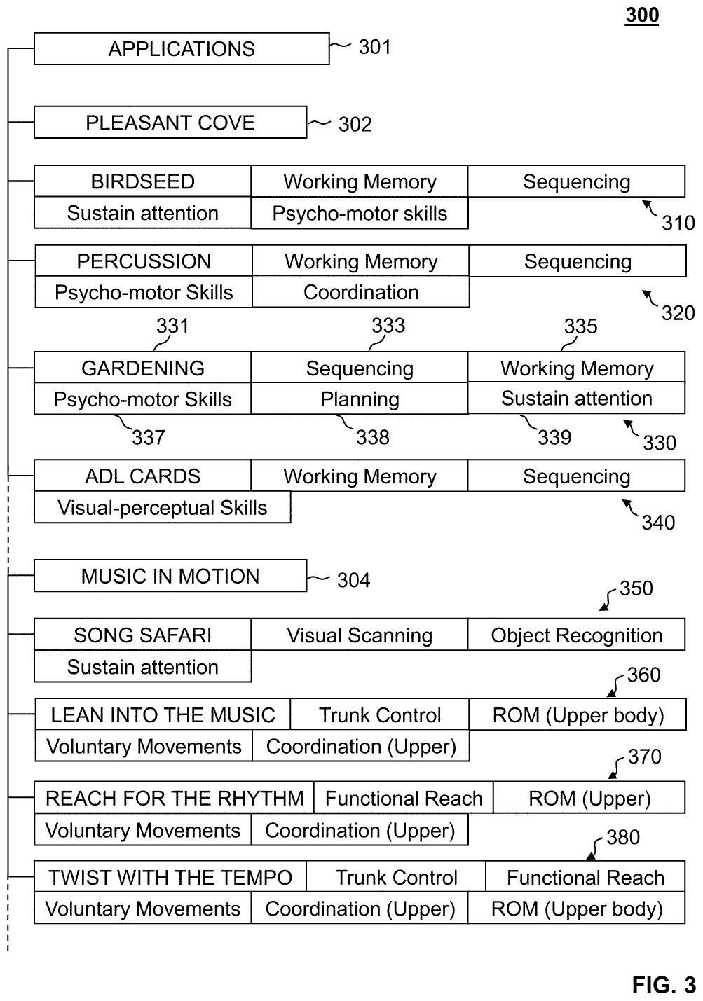

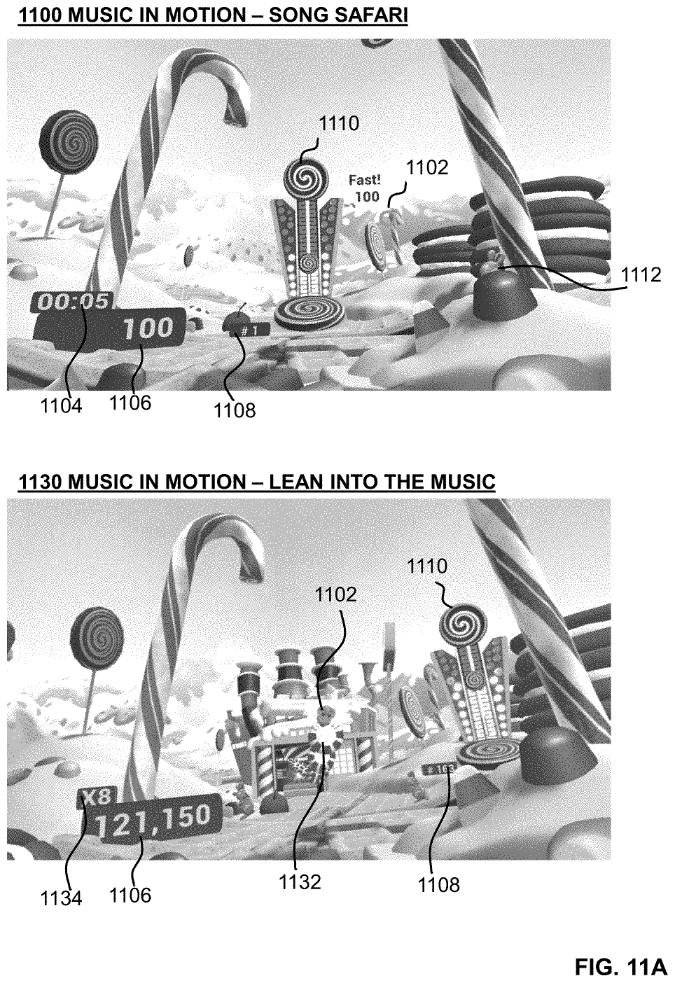

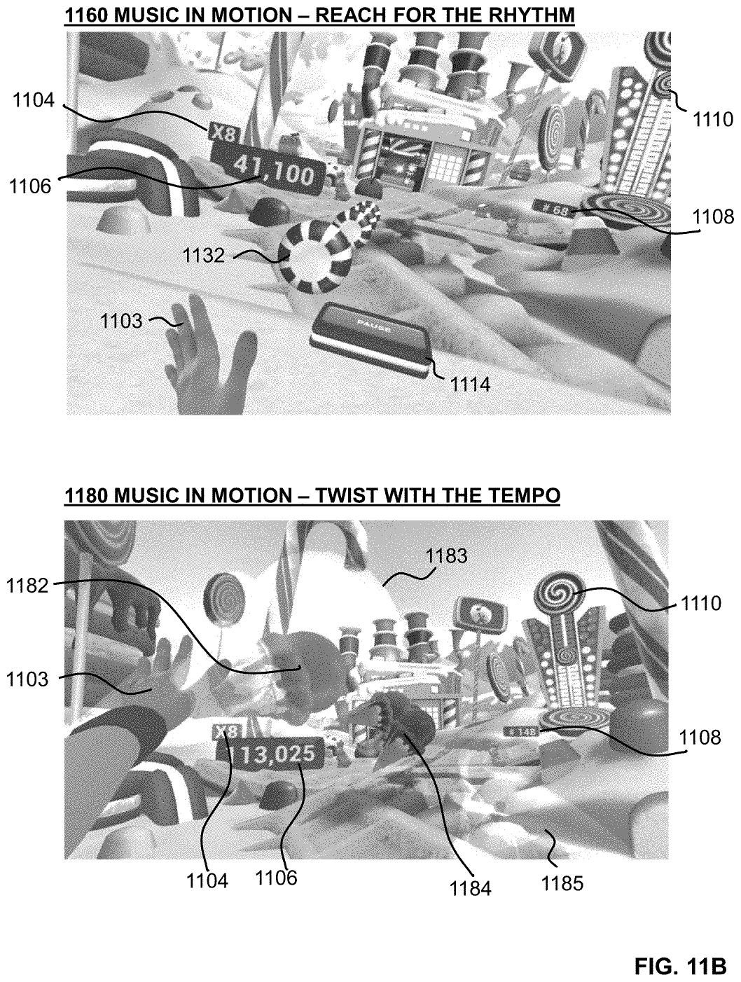

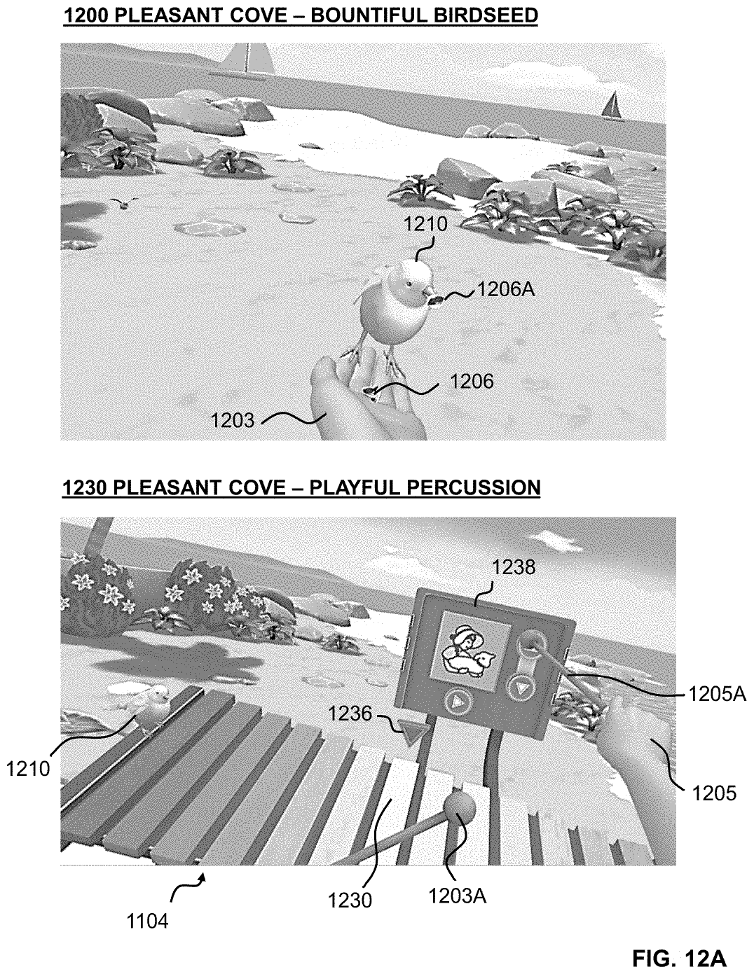

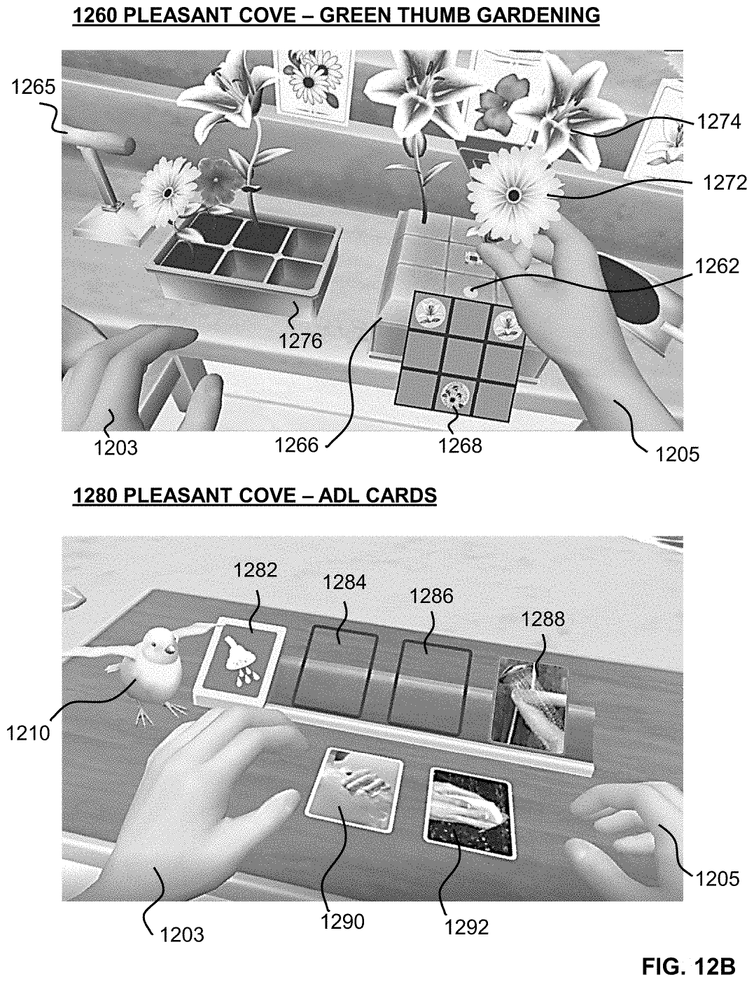

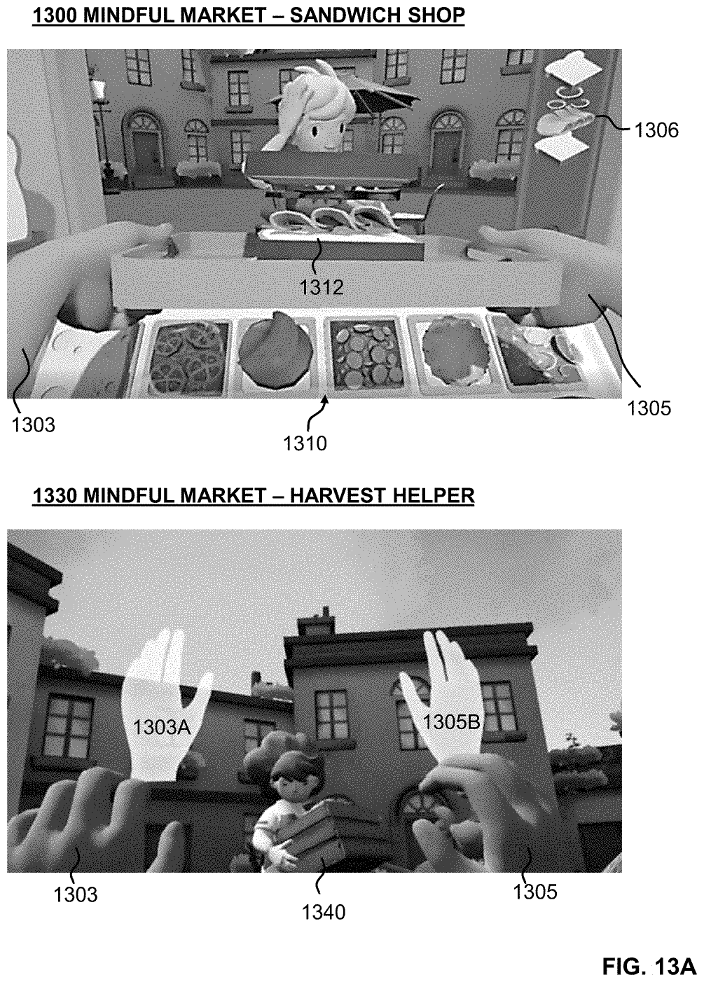

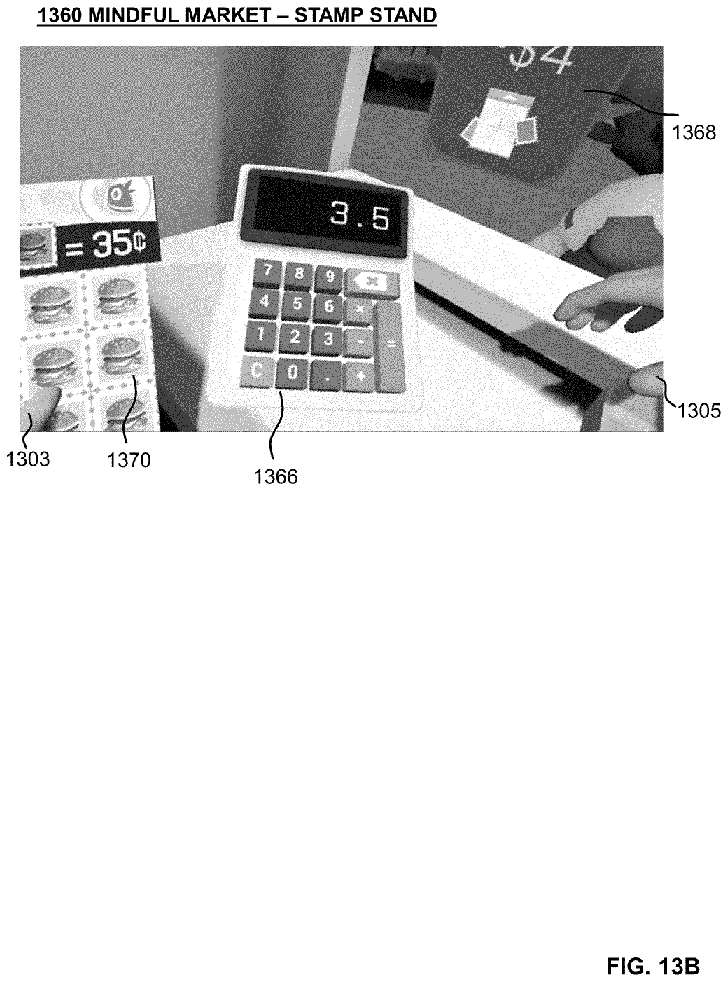

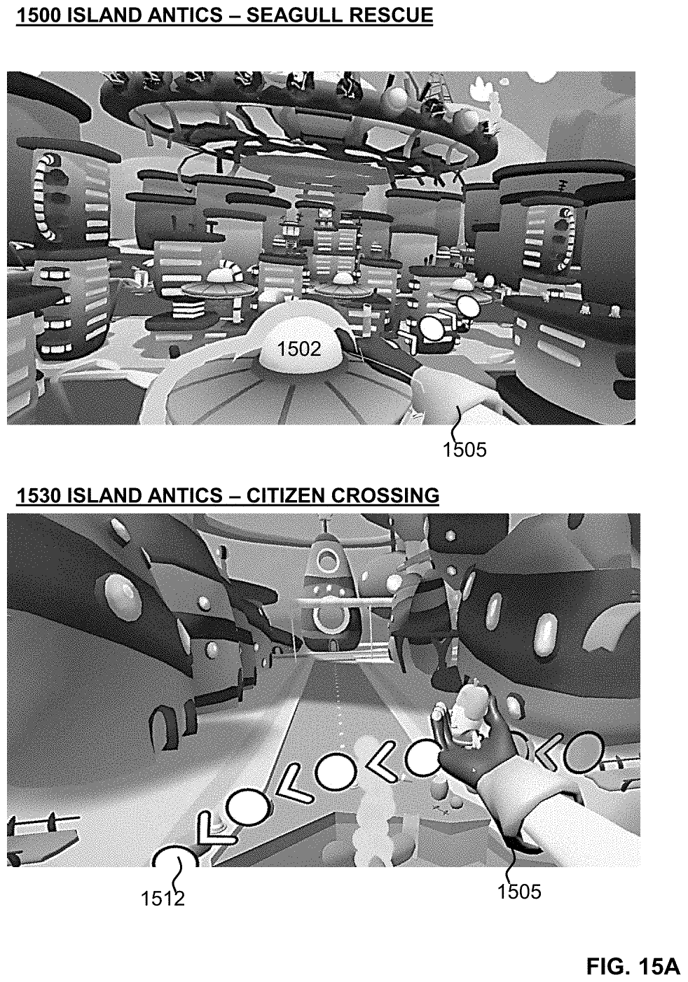

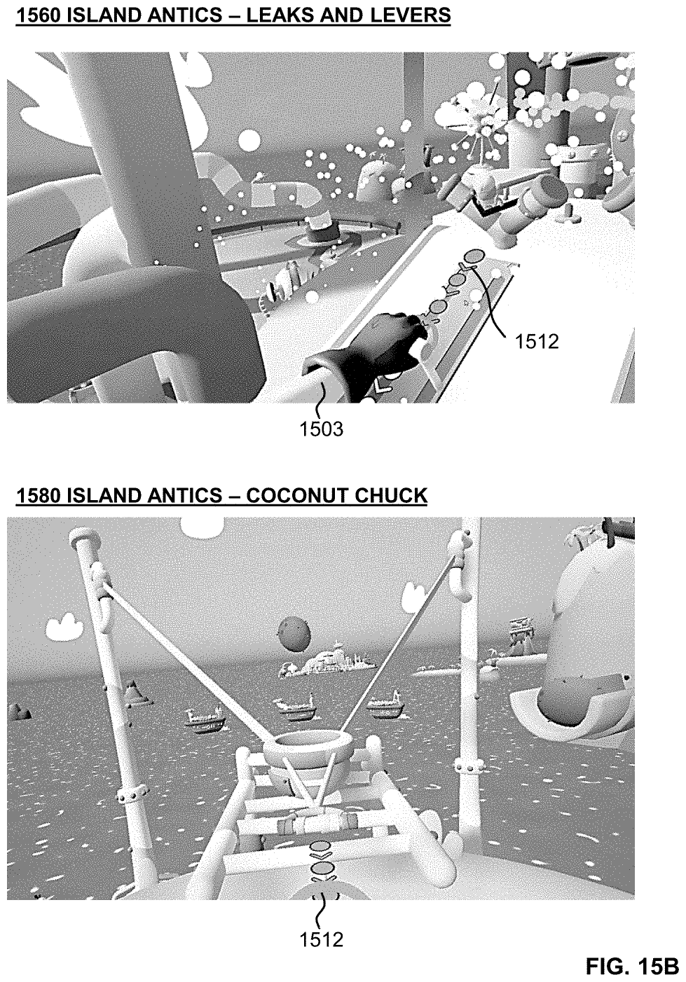

Interface 110 further depicts VR activities 122, 124, 126, and 128. In some embodiments, VR activities 122, 124, 126, and 128 may be, e.g., applications, environments, activities, games, characters, sub-activities, tasks, videos, and other content. An exemplary data structure for storing application information, including impairments that may be treated, is depicted in scenario 300 of FIG. 3. In scenario 100 of FIG. 1, in this example, VR activity 122 represents an activity from the VR application “Music in Motion,” such as an activity titled “Twist with the Tempo.” Music in Motion is depicted in FIGS. 1B, 11A, and 11B. VR activity 122 is depicted as treating, e.g., range of motion, voluntary movement, coordination, and balance. In scenario 100 of FIG. 1, VR activity 124 represents an exercise from the VR application “Island Antics,” such as the activity “Seagull Rescue.” Island Antics is depicted in FIGS. 15A and 15B. VR activity 124 is depicted as treating, e.g., trunk control, functional reach, cross-body motion, and shoulder flexion and extension. VR activity 126 may be considered to represent an activity from the VR application “Mindful Market,” such as the activity “Sandwich Shop.” Mindful Market is depicted in FIGS. 13A and 13B. VR activity 126 is depicted as treating, e.g., cognitive ability, motor control, sequencing, and working memory. In scenario 100, VR activity 128 represents a sub-activity from the VR application “Pleasant Cove,” such as the activity “Green Thumb Gardening.” Pleasant Cove is depicted in FIGS. 12A and 12B. VR activity 128 is depicted as treating, e.g., sequencing, sustained attention span, executive functioning, and working memory.

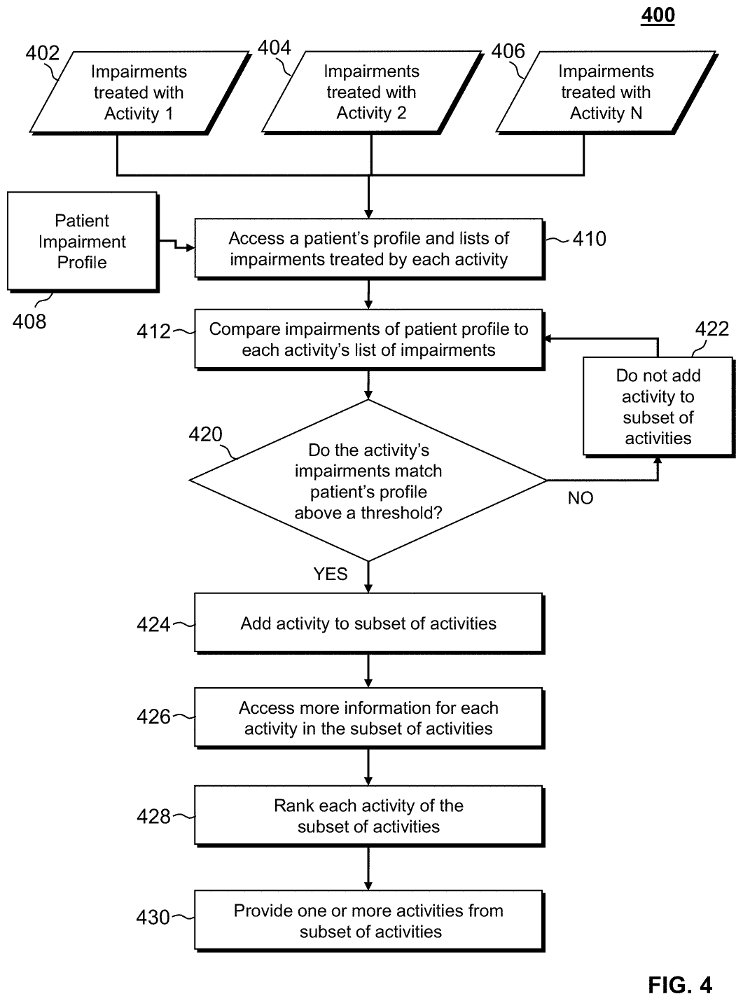

In some embodiments, VR activities 122, 124, 126, and 128 may be selected as suggested or recommended for patient profile 112. For instance, interface 110 may analyze impairments of patient profile 112 and impairments of each of the VR activities/exercises in the system to determine which activities would be most appropriate for the patient. Selecting activities to present may be accomplished in several ways. Process 400 of FIG. 4 is an exemplary process for selecting one or more activities, e.g., based on a patient's impairment profile.

FIG. 1B is an illustrative depiction of a user interface for a VR therapy platform, in accordance with some embodiments of the disclosure. By way of a non-limiting example, Scenario 150 of FIG. 1B illustrates a user interface of a VR application in a VR world as depicted to a patient view in the HMD, e.g., “Patient View.” Scenario 150 may also be considered a user interface of the same VR world as depicted to a spectator, such as a therapist, e.g., “Spectator View.”

Scenario 150 depicts, e.g., an activity from the VR application “Music in Motion,” such as an activity titled “Twist with the Tempo.” Music in Motion is also depicted in FIGS. 11A and 11B. Generally, Music in Motion is geared towards rehabilitation therapy and range-of-motion exercises using rhythm-based activities. Twist with the Tempo may be considered as a VR activity used to treat issues with, e.g., range of motion, voluntary movement, coordination, and balance. A VR system can collect patient movement data and translate it to VR avatar movement data. In some embodiments, sensors placed on the patient's body (e.g., sensors 202 as depicted in FIGS. 7B-C and 8A-C), can translate patient body movement to the VR system for animation of a VR avatar. Sensor data may also be used to measure patient movement and determine motion for patient body parts.

As depicted in scenario 150, in Twist with the Tempo, the patient is asked to reach out and touch virtual objects with the appropriate virtual hand as the objects fly by. For instance, ice cream cones 182 and 182A-D may appear to fly out of object generator 130 in time with the rhythm of a (upbeat) background song and the patient is requested to catch each object with virtual left hand 103 or virtual right hand 105. In scenario 150, ice cream cones 182 and 182A-D are designated for touching by virtual left hand 103. In some embodiments, ice cream cones may be designated by different colors and/or shapes for touching by a left or right virtual hand.

As shown in scenario 150, Twist with the Tempo also incorporates wrist turning to match each of ice cream cones 182 and 182A-D that may be rotated differently. In scenario 150, hands 103 and 105, as well as cone cursors 183 and 185, each indicate how the respective wrist is rotated, e.g., with forearm pronation and supination. One goal of Twist with the Tempo is to line up each of cone cursors 183 and 185 with incoming ice cream cones 182 and 182A-D. In some embodiments, score may be kept and how many objects are touched (by each hand) may be counted. In some embodiments, recorded performance data my incorporate scores, body movement data, range of motion, success rates, times, speed, reaction times, and other data.

In some embodiments, activities such as Twist with the Tempo may provide one or more rewards upon successful completion of a task. For example, with each object touched, a sound may be played and/or a graphic or animation may be show. Sounds may include positive-sounding noises such as a chime, bell, ring, etc. Scenario 150 also include an “excite meter” or success meter 1110. In some embodiments, a full success meter 1110 can be achieved in roughly half a song, though this can be adjusted in settings. The environment itself may indicate patient success. As success meter 1110 fills up, critters may come out to play, objects in the environment may begin to dance, and/or fireworks may light up the virtual sky. When a round, sub-activity, or activity is completed successfully, larger noises and animations may be played such as confetti, fireworks, bells, cash registers, and other sounds associated with positive reinforcement. Hearing sounds and seeing positive feedback may increase patient engagement and encourage further therapeutic progress. Generally, negative progress is not shown using the environment and success meter 1110, e.g., no lowering the meter due to failures or misses.

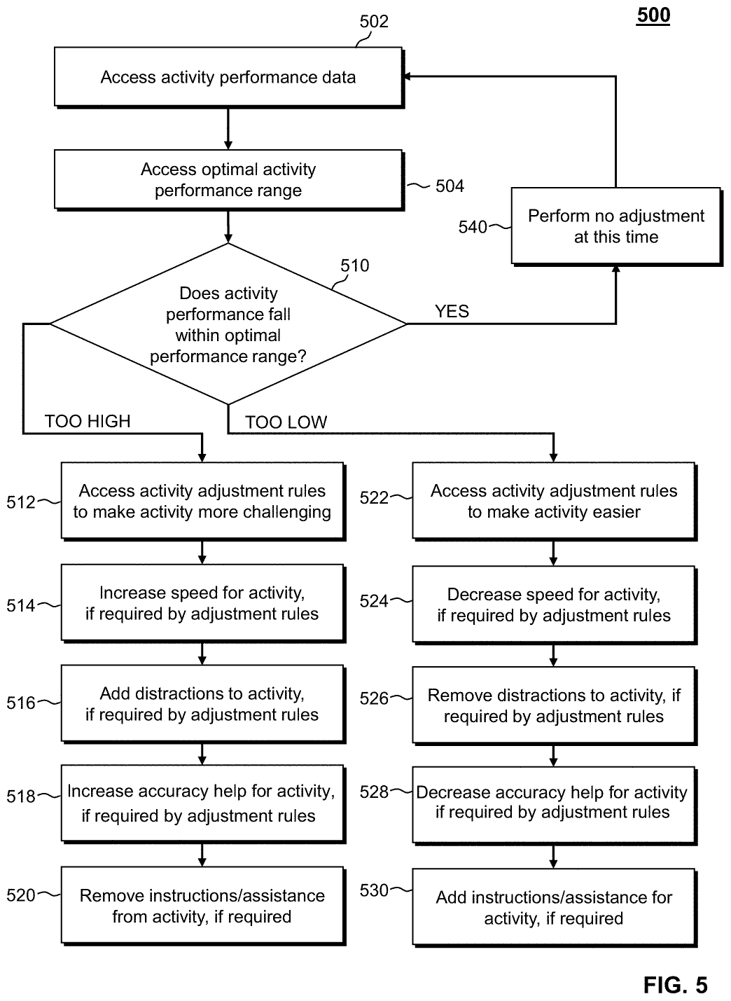

In some embodiments, activities may dynamically adjust the activity based on performance data. For instance, an activity like Twist with the Tempo may be manipulated in several ways if patient performance indicates that the activity is too easy or too difficult. Generally, performance data is based on sensor data received from a plurality of VR sensors placed on the patient's body. Performance data may be exhibited as a score or kept secretly from the patient (e.g., only viewable by the system and/or therapist). Some embodiments may use a range in the performance data as a way to maintain that a patient stays engaged. Some embodiments may have several thresholds of performance data for dynamically adjusting the exercise. Process 500 of FIG. 5 is an exemplary method of dynamically adjusting a VR activity for a patient and includes steps for determining if activity performance data falls outside the VR activity's optimal performance range and dynamically adjusting the VR activity to encourage patient engagement. Performance data may incorporate other data such as biometric feedback data. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, respiratory monitors, light sensors, cameras, sensors, and other biometric devices. Biometric feedback, along with other performance data, can indicate more subtle changes to the patient's body or physiology as well as mental state, e.g., when a patient is stressed, comfortable, distracted, tired, over-worked, under-worked, over-stimulated, confused, overwhelmed, excited, engaged, disengaged, and more. For instance, temperature sensors and infrared cameras may produce a heat map of a patient's face and determine instantaneous reactions to the activity and determine if there is a good amount of stimulation, pleasure, displeasure and/or stress.

Some embodiments may use rules to adjust the activity rules and experience when performance is too poor or too good. For instance, with Twist with the Tempo, if the patient has a higher percentage of touching objects with one hand versus the other, more objects for the weak hand may be generated. Speed or frequency of object generation may be adjusted with, e.g., more objects if performance is above the optimal range and fewer objects if performance is suboptimal. Frequency may be adjusted if metrics (e.g., heart rate, blood pressure, etc.) indicate elevated stress. With regard to the orientation of the ice creams, if performance metrics indicate that the wrist rotation is not matching well (e.g., less the 50% matches within 10% of the angle), a dynamic adjustment rule may indicate the objects should be rotated less. In some embodiments, assistance for rotation may occur to help ease the matching or better demonstrate the goal of the exercise. Size of the objects may be adjusted if performance metrics identify that a patient may not be, e.g., seeing the object. Some embodiments may provide additional guidance with activity cues, shapes, focus lights, and cursors when performance is diminished. For instance, an object may flash (more) at a time when the patient is supposed to touch it. In some cases, rules may dictate that environmental distractions such as extra animations and sounds may be limited if performance is suboptimal. For instance, bunnies, peppermint sticks, gingerbread men, etc., may not dance as much (or even appear) if the performance metrics, e.g., for eye tracking indicates performance is hindered due to too many distractions. In some embodiments, colors may change to promote more positive feelings and inspire confidence. For instance, a rule may decide that a flower object should be changed from red to yellow when metrics identify a patient may be feeling stressed, as yellow may be considered a more calming color. Rules for dynamic adjustments may be stored with application data, e.g., in exemplary data structure depicted in scenario 300 of FIG. 3, in a database, e.g., as depicted in FIG. 10.

FIG. 2 depicts an illustrative data structure for a patient profile, in accordance with some embodiments of the disclosure. Data structure 200 is an exemplary patient profile data structure for recording patient impairment data for organization and eventual comparison to treatments in VR activities. In some embodiments, a patient profile data structure may comprise a hierarchical data structure, trees, linked lists, queue, playlists, matrices, tables, blockchains, and/or various other data structures. A patient profile data structure may include, for instance, several levels of medical data, impairments, diagnoses, conditions, and linkage among similar conditions.

Profile data structure 200 depicts patient profile 112 for “Jane Doe.” Patient profile 112 includes name 232, “Jane Doe,” height/weight 234, “5′5″ 135 lbs.,” and date of birth (DOB) or age 236, “Jan. 15, 1954”. Patient profile 112 may include fields for known impairments 238, labels, areas of the body, diagnosis dates, activity performance data 290, and other relevant information such as insurance information, address, phone numbers, family information/history, and therapist notes. Patient profile 112 is shown to be documented with the patient experiencing, e.g., impairments diminishing the patient's range of motion (condition 240), trunk control (condition 250), functional reach (condition 260), executive functioning (condition 270), sequencing, and working memory. In condition 240, label 241 indicates “range of motion,” primary area 247 indicates “trunk,” secondary area 248 indicates “left shoulder,” first diagnosis date 243 indicates Mar. 5, 2017, and latest diagnosis date 245 indicates Apr. 9, 2021. Executive functioning of condition 270 in FIG. 2 includes conditions (or sub-conditions) sequencing, working memory, and self-control. In same embodiments, sequencing, working memory, and self-control may each be separate conditions in profile 112 or may be linked based on diagnosis, similarities in conditions, and/or other connections. Patient profile 112 may include further impairment data, health data, biometric data, VR activity data, and other relevant data. A portion of exemplary data structure patient profile 112 is depicted in scenario 100 of FIG. 1. In some embodiments, each condition may be ranked, prioritized, tiered, or otherwise weighted to signify importance in comparison to other conditions. For instance, an issue with range of motion (condition 240) may be more severe than an issue with self-control (e.g., part of condition 270).

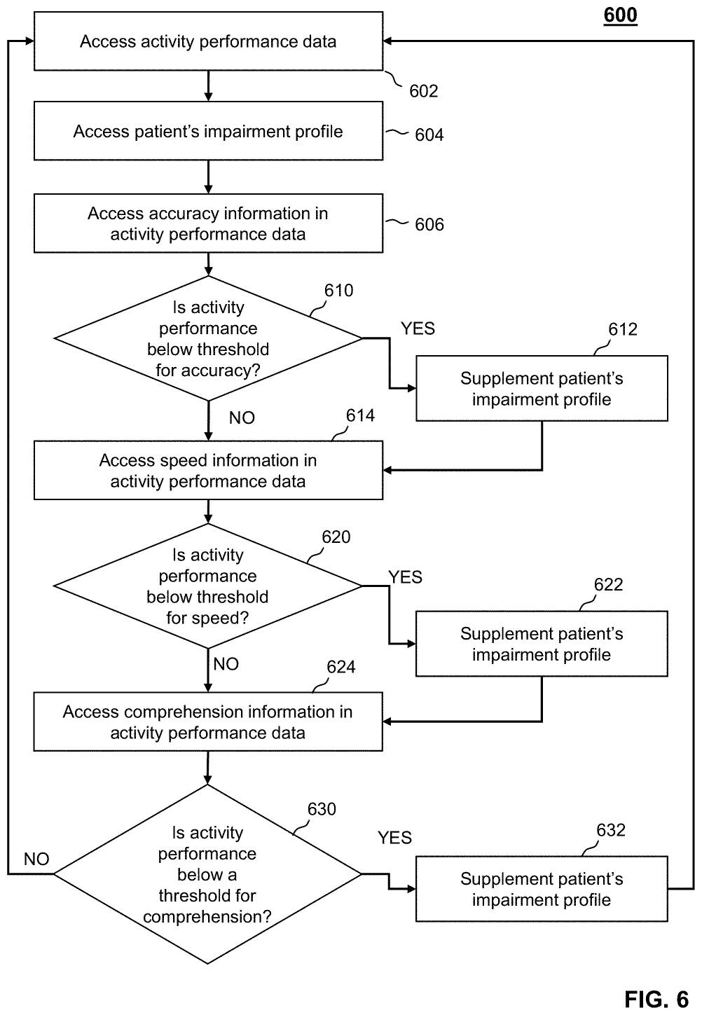

In some embodiments, patient profile 112 may include fields for detected potential impairments 239. For instance, if activity performance data indicates a potential impairment, a new condition may be added to the potential impairments 239 section of profile 112. In data structure 200, detected potential impairments 239 includes condition 280, “eye-hand coordination” which is identified in the left hand and, e.g., first noted on the date “6/6/2021.” For instance, a VR therapy session on that date may have yielded performance data that was below a threshold associated with eye-hand coordination and noted on a specific first date. Process 600 of FIG. 6 is an exemplary process for identifying potential impairments based on a patient's performance in a VR activity.

In some embodiments, patient profile 112 may include activity performance data 290. For instance, activity performance data 290 may include activity logs and performance metrics such as times, scores, repetitions, difficulty, range of motion, and other measurements.

Patient profile 112 may be accessed and loaded, e.g., as a patient logs in to a VR therapy platform or application. In some embodiments, loading patient profile 112 may be initiated by a therapist or supervisor. Patient profile 112 may be stored in a secure database, e.g., as depicted in FIG. 10, and only accessed by the appropriate patient and clinicians, so as to minimize risk of violating any privacy laws or codes of ethics. In some embodiments, a patient profile data structure may be stored in or with a VR user profile, e.g., at a server. In some embodiments, a patient profile data structure may be stored, for instance, at an encrypted cloud server. Moreover, in some embodiments, a patient profile data structure may be stored locally at the device. For instance, a patient profile may need to be kept private, e.g., encrypted and stored only at one device.

FIG. 3 depicts an illustrative data structure for VR applications and activities, in accordance with some embodiments of the disclosure. Data structure 300 is an exemplary VR therapeutic activity data structure for managing associated impairment data to be compared to patient profiles for matching. In some embodiments, an activity data structure may comprise a hierarchical data structure, trees, linked lists, queue, playlists, matrices, tables, blockchains, and/or various other data structures. An activity data structure may include, for instance, several levels of activity data, tasks, rules, impairments, thresholds, conditions, and linkage among similar conditions.

Data structure 300 comprises a list of applications 302 including exemplary applications such as Pleasant Cove 302 and Music in Motion 304. Data structure 300 may comprise many more applications, e.g., dozens or hundreds, and may be updated routinely as application and activity offerings are updated, added, and/or removed within the platform. In some embodiments, exemplary applications Pleasant Cove 302 and Music in Motion 304 may be referred to as worlds, settings, activities, etc. Pleasant Cove is depicted in FIGS. 12A and 12B and Music in Motion is depicted in FIGS. 1B, 11A, and 11B. Within each application of application 301, there are activities, such as activities 310, 320, 330, 340, 350, 360, 370, and 380. In some embodiments, activities may be referred to as sub-activities, exercises, tasks, or other similar characterizations. Exemplary activity 330 is depicted with title 331 (“Gardening”) that may be considered as referring to Green Thumb Gardening of Pleasant Cove, depicted in FIGS. 12A and 12B. Exemplary activity 330 is also associated with conditions that may be treated by the activity, e.g., condition 333 (sequencing), condition 335 (Working Memory), condition 337 (psycho-motor skills), condition 338 (planning), condition 329 (sustain attention). Within application Pleasant Cove 302 are activity 310 (Birdseed, e.g., Bountiful Birdseed), activity 320 (Percussion, e.g., Playful Percussion), activity 330 (Gardening), and activity 340 (ADL Cards). Within application Music in Motion 304 are activity 350 (Song Safari), activity 360 (Lean into the Music), activity 370 (Reach for the Rhythm), and activity 380 (Twist with the Tempo). Each activity has at least one condition to be treated associated with it.

In some embodiments, conditions associated with each activity may be weighted or prioritized by focus. For instance, in activity 350, Song Safari, the activity may focus more on visual scanning than sustaining attention. Based on prioritization, exercising a patient's working memory may be focus on more in activity 320, Percussion, than in activity 330, Gardening. In some embodiments, each condition may be given a score (e.g., 1-100) or percentage weight based on its use in the activity. Data structure 300 may store weights and prioritization scores for conditions within each activity.

FIG. 4 depicts an illustrative flowchart of a process for selecting an appropriate VR activity for a patient, in accordance with some embodiments of the disclosure. There are many ways to identify appropriate VR activities and sub-activities for treating a patient and process 400 is one example. Generally, process 400 of FIG. 4 includes steps for comparing impairments of a patient's profile to each activity's list of impairments to be treated, determining if the impairments match, e.g., above a threshold, and providing a subset of activities matching the patient's impairments.

Some embodiments may utilize a VR engine to perform one or more parts of process 400, e.g., as part of a VR application, stored and executed by one or more of the processors and memory of a headset, server, tablet and/or other device. For instance, VR engine may be incorporated in one or more of head-mounted display 201 and clinician tablet 210 of FIGS. 7A-D and/or the systems of FIGS. 9-10. A VR engine may run on a component of a tablet, HMD, server, display, television, set-top box, computer, smartphone, or other device.

At step 402, a VR engine receives a list of impairments able to be treated with Activity 1. For instance, Activity 1 may be considered an activity titled “Twist with the Tempo” from the VR application “Music in Motion,” depicted in FIGS. 1B, 11A, and 11B. The list of impairments associated with Activity 1, e.g., conditions that may be treated by Activity 1, may be stored with application and activity data in a database, e.g., as depicted in FIG. 10. An exemplary data structure for storing application information, including impairments that may be treated, is depicted in scenario 300 of FIG. 3. In some embodiments, Activity 1, “Twist with the Tempo,” may treat impairments with, e.g., range of motion, voluntary movement, coordination, functional reach, and balance, among other conditions. In some embodiments, impairments treated by an exercise may be prioritized or weighted based on prevalence within the activity. For instance, an activity's focus on improving range of motion may be a tier 1 impairment (e.g., weighted at 100%) while balance may be a tier 2 or less focused on impairment (e.g., weighted at 80%). Different activities may have different scores or weights for various impairments.

At step 404, the VR engine receives a list of impairments able to be treated with Activity 2. For instance, Activity 2 may be considered an activity titled “Seagull Rescue” from the VR application “Island Antics,” as depicted in FIGS. 15A and 15B. Activity 2, Seagull Rescue, may treat impairments such as, e.g., trunk control, functional reach, cross-body motion, and shoulder flexion and extension, among other conditions.

At step 406, the VR engine receives a list of impairments able to be treated with Activity N. In some embodiments, Activity N may represent the last of N activities available. Some embodiments may feature a handful of activities, while some other embodiments may include dozens or more VR applications and/or activities. For instance, Activity N may be considered an activity titled “Green Thumb Gardening” from the application “Pleasant Cove.” Pleasant Cove is depicted in FIGS. 12A and 12B. Activity N, “Green Thumb Gardening,” may treat impairments such as, e.g., sequencing, sustained attention span, executive functioning, and working memory, among other conditions.

At step 408, the VR engine receives a list of impairments from a patient's impairment profile. For instance, a patient may be participating in VR therapy and her profile is prepared for access by the VR engine. In scenario 100, patient profile 112 for “Jane Doe” is received. A patient, for example, may be experiencing difficulty or discomfort with, e.g., range of motion, trunk control, functional reach, executive functioning, sequencing, and working memory. Patient profiles may be stored in a secure database, e.g., as depicted in FIG. 10. Like in scenario 200, the received patient profile may be considered to list impairments such as range of motion in trunk and left shoulder, limited control of the trunk, issues with functional reach affecting the left arm and left shoulder, and limitations with executive functioning, sequencing, working memory, and self-control. In some embodiments, impairments in a profile may be prioritized or weighted based on the patient's needs. For instance, a patient's trunk control issue may be a tier 1 impairment (e.g., weighted at 100%) while her functional reach with the left arm may be a tier 2 or lower impairment (e.g., weighted at 80%). In some embodiments, a patient profile may be received prior to or when a patient logs into the system and/or begins a therapy session.

At step 410, the VR engine accesses the patient's profile and the lists of impairments treated by each activity. Patient profiles, for instance, may be stored in a secure database, e.g., as depicted in FIG. 10.

At step 412, the VR engine compares the impairments identified in the patient profile to each activity's list of impairments to be treated. In some embodiments, impairments from each activity's list and the patient profile may be compared. In some embodiments, matches are identified and counted. In some embodiments, matches from each activity's list may be prioritized or weighted based on prevalence within the activity or in the patient profile. For instance, matches are identified and weighted based on a tier of each impairment (e.g., prioritization). A match of an activity prioritizing trunk control for a patient with significant trunk control issues may be weighted (e.g., 125%) more than a match with an activity focusing on working memory when the patient has only minor memory issues (e.g., 50%). Comparisons may be performed in several ways. In some embodiments, impairments from each activity's list and the patient profile may be correlated and a match score (e.g., 1-100) for the activity calculated. For instance, each impairment of a patient profile and each VR activity may be given a numeric identifier and a weight value. In some embodiments, numeric identifiers and each corresponding weight value for a profile or VR activity may form matrices and the matrices are correlated. In some embodiments, numeric identifiers and each corresponding weight value for a profile or VR activity may be charted as coordinates and use linear regression to compare. In some embodiments, an index of every impairment treatable by all the applications may be used, wherein each impairment is associated with one or more applications and/or activities that may treat the impairment or condition.

In some embodiments, a comparison may be made by a trained model using, e.g., a neural network. For instance, a model may be trained to accept a patient profile as input and identify one or more VR activities suitable for the patient profile. Such a model may be trained by doctors and/or therapists who prove training data of profiles and identify which VR activities may be appropriate for use. Then, using a feedback loop, the model can be further trained with test patient profiles by, e.g., rewarding the neural network for correct predictions of suitable VR activities and retraining with incorrect predictions. In some embodiments, a comparison may use a combination of a trained model and comparative analysis.

At step 420, for each activity, the VR engine determines whether the activity's treated impairments match the impairments identified in the patient's profile, e.g., above a predetermined threshold. For instance, if the counted matches between an activity and the patient profile meet or exceed a threshold (e.g., five matches), the activity may be further analyzed. In some embodiments, e.g., when a weighting or correlation is used, the threshold may be a score from 1-100, such as 75, and the activity match score must meet or exceed the match score threshold. In some embodiments, the threshold may be based on the number of impairments in a profile, e.g., the threshold may be two-thirds (66%) of the total number of impairments in a profile. In some embodiments, the match threshold may be one match, e.g., in situation where patients have only one or a couple impairments.

If the VR engine determines an analyzed activity's treated impairments do not match the impairments identified in the patient's profile above a threshold, then, at step 422, the VR engine does not add activity to a subset of activities for further analysis. For instance, if the threshold is five matches and the activity only has two matches, the activity is discarded for now. In some embodiments, if the threshold is a match score of 75 and the activity only has a match score of 40, the activity is discarded for now. In some embodiments, if all the activities are evaluated for matches and none meet the predetermined threshold, a second (lower) predetermined threshold may be used (e.g., half the first threshold).

If the VR engine determines an activity's treated impairments do match the impairments identified in the patient's profile above a threshold, then, at step 424, the VR engine adds the matching activity to a subset of activities. For instance, if the threshold is four matches and the activity has six matches, the activity is added to the subset for further review. In some embodiments, if the threshold is a match score of 85 and the activity has a match score of 92, the activity is added to the subset for further review.

At step 426, the VR engine accesses more information for each activity of subset of activities. For instance, additional information for an activity may comprise warnings of impairments that should not attempt the activity, calendar data of when the activity was last accessed, compatibility data, activity version and update data, average activity duration data, activity performance data, and other data. For instance, some additional activity data may indicate recent participation in an activity and/or recent success/struggles with the activity. In some embodiments, additional information may include recommendation/weighting by a doctor or therapist indicating a preference to use (or not use) a particular motion required by one or more activities. In some embodiments, an activity may be eliminated from the subset if, e.g., a conflict arises based on additional activity data. In some embodiments, a warning of a potential conflict may be provided.

At step 428, the VR engine ranks each activity of the subset of activities. For instance, the VR engine may rank each activity of the subset of activities based on a match count or a match score. In some embodiments, the VR engine may weight a match count or a match score differently based, e.g., on the activity's additional information. For instance, an activity's match count (or score) may decrease if there is significant focus on an exercise, e.g., cross-body motion, that may be too difficult to perform with another impairment (e.g., balance). In some embodiments, the VR engine may adjust rankings of similarly scoring activities based on recent performance of the activity and/or recent success/struggles with the activity.

At step 430, the VR engine provides one or more activities from the subset of activities. For instance, scenario 100 of FIG. 1 depicts a menu for a VR therapy platform suggesting VR activities 122, 124, 126, and 128 based on an identified patient profile 112. In scenario 100, the matches between profile 112 and each of activities 122, 124, 126, and 128 are apparent. Patient profile 112 for “Jane Doe” indicates difficulty or discomfort with, e.g., range of motion, trunk control, functional reach, executive functioning, sequencing, and working memory. Activity 1 from step 402, Twist with the Tempo (VR activity 124 of FIG. 1), may treat impairments with, e.g., range of motion, voluntary movement, coordination, functional reach, and balance, among other conditions. Activity 2 from step 404, Seagull Rescue (VR activity 124 of FIG. 1), may treat impairments such as, e.g., trunk control, functional reach, cross-body motion, and shoulder flexion and extension, among other conditions. Activity 1 may be ranked higher than Activity 2 because there are more matches. In some embodiments, Activity 1 may be ranked ahead of Activity 2 because Activity 2 requires movement that may adversely impact the patient, in accordance with data in the patient profile. Activity N from step 406, Green Thumb Gardening, may treat impairments such as, e.g., sequencing, sustained attention span, executive functioning, and working memory, among other conditions. While Activity N from step 406, Green Thumb Gardening, may have some matches with the Jane Doe profile, the matches do not merit ranking as high as, e.g., Activity 1 (Twist with the Tempo) or Activity 2 (Seagull Rescue).

FIG. 5 depicts an illustrative flowchart of a process for dynamically adjusting a VR activity for a patient, in accordance with some embodiments of the disclosure. There are many ways to dynamically adjusting a VR activity for a patient and process 500 is one example. Generally, process 500 of FIG. 5 includes steps for determining if activity performance data falls outside the VR activity's optimal performance range and dynamically adjusting the VR activity to encourage patient engagement. Some embodiments may utilize a VR engine to perform one or more parts of process 500, e.g., as part of a VR application, stored and executed by one or more of the processors and memory of a headset, server, tablet and/or other device and/or the systems, e.g., from FIGS. 7A-D and FIGS. 9-10.

At step 502, a VR engine accesses activity performance data. For instance, in activities like Twist the Tempo depicted in FIGS. 1B and 11B, performance data may include score, object count, streak counts, hand and arm position data, head/eye position data, etc. In some embodiments, recorded performance data my incorporate score rates, body movement data, range of motion, success rates, times, speed, reaction times, and other data. Performance data may be exhibited as a score or kept secretly from the patient (e.g., only viewable by the system and/or therapist). In some embodiments, activity performance data may comprise additional biometric feedback. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, respiratory monitors, light sensors, cameras, sensors, and other biometric devices. Biometric feedback, along with other performance data, can indicate more subtle changes to the patient's body, e.g., when a patient is stressed, comfortable, distracted, tired, over-worked, under-worked, over-stimulated, confused, overwhelmed, excited, engaged, disengaged, and more. Performance data, including scores, records, biometric may be stored with profile and/or application data, e.g., in exemplary data structures depicted in scenarios 200 and 300 of FIGS. 2 and 3, respectively, in a secure database, e.g., as depicted in FIG. 10. Some embodiments may include a VR impairment assessment activity that specifically tests a patient for one or more impairments.

At step 504, the VR engine accesses optimal activity performance range for the activity. For instance, in activities like Twist the Tempo, an ideal object hit rate may be around 65-85%. In some embodiments, an optimal performance range may be a heart rate of, e.g., 50-75% of a patient's maximum heart rate, as indicated in the patient profile (e.g., estimated by subtracting the patient's age from 220). For instance, Jane Doe may have a max heart rate of 153, which would make an optimal range for heart rate to be 76-115. Some embodiments may track eye movement and/or head direction and measure time spent focused on background stimuli and a proper range may be, e.g., 10-20% of the activity time. Some embodiments may track face, skin, and/or brain temperature and monitor whether the portions have a temperature that falls outside of the range of, e.g., 98-99° F.

At step 510, the VR engine determines if the activity performance falls within optimal performance range. For instance, in activities like Twist the Tempo, an ideal object hit rate may be around 65-85%, with performance below 65% indicating a need for help and performance above 85% indicating a need for further challenge. Some embodiments may only look at activity performance ranges based on the patient's profile, e.g., a patient's impairments. For instance, if a patient is experiencing impairment with trunk movement, the VR engine may discard performance ranges for unrelated portions of an activity in question.

If the VR engine determines the activity performance is higher than the optimal performance range then, at step 512 the VR engine accesses activity adjustment rules to make activity more challenging. For instance, in Twist the Tempo, performance above 85% may indicate a need for further challenge under the activity adaptation rules. In some embodiments, additional challenges may be a series of steps to push the patient to perform at a higher level. For instance, with Twist with the Tempo, if a successful patient has a higher percentage of touching objects with one hand versus the other, more objects for the weak hand may be generated. Speed or frequency of object generation may be adjusted with, e.g., more objects if performance is above the optimal range.

At step 514, the VR engine increase speed for the activity, if required by the activity's adjustment rules. For instance, with Twist with the Tempo, the music may speed up and more ice cream cones may appear, e.g., for both digital hands to touch.

At step 516, the VR engine adds more distractions to the activity, if required by the activity's adjustment rules. For instance, with Twist with the Tempo, the background may become more animated, or the sounds may become a bit more distracting as the patient has success. If successful in Twist with a Tempo, critters may come out to play, objects in the environment may begin to dance, and/or fireworks may light up the virtual sky.

At step 518, the VR engine increases the required accuracy in the activity, if required by the activity's adjustment rules. For instance, with Twist with the Tempo, matching the angles of the ice cream cones as they fly towards the patient may have a tolerance of, e.g., 10 degrees in either direction. If performance data indicates success above the optimal range, the tolerance for the rotation matching may be thinned down to 5 degrees in either direction. In activities where a ball or rock may fall into a basket or bucket when generally close, if performance data indicates success above the optimal range (e.g., 70-90%), the accuracy threshold may become more difficult. In activities where a flower must be placed on a mark, guidance for directing the flower into the right spot may no longer be provided, e.g., when performance is faster than the optimal range (e.g., 5-15 seconds).

At step 520, the VR engine removes or limits instructions for and/or assistance from the activity, if required by the activity's adjustment rules. For example, in Playful Percussion of Pleasant Cove (FIGS. 12A and 12B), if the success rate is above the optimal range, the correct xylophone key may not light up more than once and/or the cursor might not appear immediately. In some activities, such as Pleasant Cove, a non-playable character such as a bird or squirrel guides the patient in the exercise. In activities that may limit guidance when above-optimal scores are achieved, the guide may not be as talkative or helpful.

If the VR engine determines if the activity performance is lower than the optimal performance range then, at step 522, the VR engine accesses activity adjustment rules to make activity easier. Some embodiments strive to better engage struggling patients and inspire patients to continue to work and follow through in their therapy. No one wants to discourage patient participation and dynamically making an activity a bit easier while a patient develops, may help draw the patient into the experience more. For instance, in Twist the Tempo, performance below 65% may indicate a need for further help under the activity adaptation rules. In some embodiments, reducing stress-inducing challenges may be a series of steps to encourage the patient to perform at a therapeutic level. For instance, with Twist with the Tempo, if a struggling patient has a lower percentage of touching objects with one hand versus the other, more objects for the strong hand may be generated until the weaker hand improves. Speed or frequency of object generation may be adjusted with, e.g., fewer objects flying at a slower pace if performance is below the optimal range.

At step 524, the VR engine decreases speed for activity, if required by the activity's adjustment rules. For instance, with Twist with the Tempo, the music may slow down, and fewer ice cream cones may appear, e.g., for both digital hands to touch. In some cases, a countdown timer may be suspended or slowed. In some activities, animations may be decelerated until the patient improves performance and/or metrics indicate less stress.

At step 526, the VR engine removes distractions to the activity, if required by the activity's adjustment rules. For instance, with Twist with the Tempo, the background may become less animated, or the sounds may become less distracting as the patient has struggles. If struggling in Twist with a Tempo, critters may no longer dance in the background, objects in the environment may disappear, and/or fireworks may be eliminated or delayed if the performance metrics, e.g., for head/eye tracking indicates performance is hindered due to too many distractions. In some activities, such as flower arranging activities, a rule may decide that a flower object should be changed from red to yellow when metrics identify a patient may be feeling stressed, as yellow may be considered a more calming color.

At step 528, the VR engine decreases required accuracy for the activity, if required by the activity's adjustment rules. For instance, with Twist with the Tempo, matching the angles of the ice cream cones as they fly towards the patient may have a tolerance of, e.g., 10 degrees in either direction. If performance data indicates a patient performs below the optimal range, the tolerance for the rotation matching may be widened to, e.g., 15 or 20 degrees in either direction. In activities where a ball or rock may fall into a basket or bucket when generally close, if performance data indicates performance below the optimal range (e.g., 55-85%), the accuracy threshold may become easier. In activities where a flower must be placed on a mark, guidance for directing the flower into the right spot may be provided and automatically pulled in, e.g., when performance is slower than the optimal range (e.g., 5-15 seconds).

At step 530, the VR engine may add instructions for and/or assistance from the activity for activity, if required by the activity's adjustment rules. For example, in Playful Percussion of Pleasant Cove (FIGS. 12A and 12B), if the success rate is below the optimal range, the correct xylophone key may light up more frequently and/or the cursor might appear quicker, bounce, and or/be a brighter color. In some activities, such as Pleasant Cove, a non-playable character such as a bird or squirrel may guide the patient in the activity. In activities that may add guidance when below-optimal scores are performed, the guide may much more talkative or helpful.

If the VR engine determines if the activity performance is higher than the optimal performance range then, at step 540, no adjustment is performed at this time and further activity performance data is accessed at step 502, e.g., to restart process 500. In some embodiments, these dynamic adjustments are temporary and return to normal when, e.g., performance falls within the range or a time limit expires or the activity is exited or completed.

FIG. 6 depicts an illustrative flowchart of a process for identifying potential impairments based on patient performance in a VR activity, in accordance with some embodiments of the disclosure. There are many ways to identifying potential impairments based on a patient's performance in a VR activity and process 600 is one example. Generally, process 600 of FIG. 6 includes steps for determining if particular performance data falls below a threshold for accuracy, speed, and/or comprehension in a VR activity and supplementing a patient's impairment profile, e.g., as a potential impairment, if a threshold is not met. Some embodiments may utilize a VR engine to perform one or more parts of process 600, e.g., as part of a VR application, stored and executed by one or more of the processors and memory of a headset, server, tablet and/or other device and/or the systems, e.g., from FIGS. 7A-D and FIGS. 9-10.

At step 602, a VR engine accesses activity performance data. For instance, in activities like Twist the Tempo depicted in FIGS. 1B and 11B, performance data may include score, object count, streak counts, hand and arm position data, head/eye position data, etc. In some embodiments, recorded performance data my incorporate score rates, body movement data, range of motion, success rates, times, speed, reaction times, and other data. In some embodiments, activity performance data may comprise additional biometric feedback. Biometric feedback, along with other performance data, can indicate more subtle changes to the patient's body, e.g., when a patient is stressed, comfortable, distracted, tired, over-worked, under-worked, over-stimulated, confused, overwhelmed, excited, engaged, disengaged, and more. Performance data, including scores, records, biometric may be stored with profile and/or application data, e.g., in exemplary data structures depicted in scenarios 200 and 300 of FIGS. 2 and 3, respectively, in a secure database, e.g., as depicted in FIG. 10.

At step 604, the VR engine accesses a patient's impairment profile. For instance, a patient may be participating in VR therapy and her profile is prepared for access by the VR engine. In scenario 100, patient profile 112 for “Jane Doe” is received. A patient, for example, may be experiencing difficulty or discomfort with, e.g., range of motion, trunk control, functional reach, executive functioning, sequencing, and working memory. Patient profiles may be stored in a secure database, e.g., as depicted in FIG. 10. An exemplary data structure for storing patient profile 112 is depicted in scenario 200 of FIG. 2. Like in scenario 200, the accessed patient profile may be considered to list impairments such as range of motion in trunk and left shoulder, limited control of the trunk, issues with functional reach affecting the left arm and left shoulder, and limitations with executive functioning, sequencing, working memory, and self-control.

At step 606, the VR engine analyzes accuracy in activity performance data in view of impairment profile. For instance, in activities like Twist the Tempo, accuracy may include percentage of objects touched, as well as percentage of touches matching the correct rotation. In Playful Percussion, accuracy may be counts of correct and incorrect notes played. In Green Thumb Gardening or Sandwich Shop, accuracy may measure counts for correct or incorrect sequencing. In Citizen Crossing of Island Antics, depicted in FIG. 15A, accuracy may be a measurement of how closely each hand/arm follows the path.

At step 610, the VR engine determines if the activity performance data is below a predetermined threshold for accuracy. For instance, performance may be subpar in Twist the Tempo, if accuracy of objects touched is below a threshold of 35%, or the percentage of touches matching the correct rotation is below, e.g., 50%. In Playful Percussion, if accuracy of counts of correct notes is below 50% or incorrect notes played is, e.g., above 80% accuracy may be an issue. In Green Thumb Gardening or Sandwich Shop, counts for correct sequence below 50% may indicate an issue. In Citizen Crossing, if a measurement of how closely each hand/arm follows the path is below 25%, there could be an accuracy issue.

If the VR engine determines the activity performance is lower than the predetermined threshold for accuracy then, at step 612, the patient's impairment profile is supplemented. For instance, the VR engine may supplement a patient's impairment profile, e.g., as a potential impairment, if a threshold is not met with one or more associated impairments. With the potential impairment identified, doctors and therapists may further examine and test for the impairment. For instance, in Twist the Tempo, if accuracy of objects touched is below a threshold of 35%, or the percentage of touches matching the correct rotation is below, e.g., 50%, there may be issues with functional reach, coordination, and/or control. In Playful Percussion counts of correct notes is below 50% and incorrect notes played is, e.g., above 80%, there may be issues with, e.g., working memory and/or sequencing. In Green Thumb Gardening or Sandwich Shop, counts for correct sequence below 50% may indicate an issue with sequencing, there may be issues with short-term working memory. In Citizen Crossing, if a measurement of how closely each hand/arm follows the path is below 25%, there could be an issue with functional reach and/or coordination.

At step 614, the VR engine analyzes speed in activity performance data in view of impairment profile. For instance, in Playful Percussion, speed may be measured as correct and incorrect notes played during the duration of the song being played. In Green Thumb Gardening or Sandwich Shop, time and rate of project completion may be a measurement of speed. In Citizen Crossing, speed may be a measurement of how long it takes for each hand/arm gesture as it follows the path to move/rescue the non-playable characters.

At step 620, the VR engine determines if the activity performance data is below a predetermined threshold for speed. For instance, in Playful Percussion, if duration between correct notes is above 45 seconds, speed performance may be an issue. In Green Thumb Gardening or Sandwich Shop, if duration is consistently too long (e.g., greater than 3 minutes), speed may be an issue. In Citizen Crossing, if each rescue takes longer than 45 seconds, there could be a speed issue.

If the VR engine determines the activity performance is lower than the predetermined threshold for speed then, at step 622, the patient's impairment profile is supplemented. For instance, in Playful Percussion, if the duration between correct notes is above 45 seconds, there may be issues with, e.g., working memory and/or sequencing. In Green Thumb Gardening or Sandwich Shop, if duration is consistently too long (e.g., greater than 3 minutes) there may be issues with short-term working memory. In Citizen Crossing, if each rescue takes longer than 45 seconds, there could be an issue with functional reach and/or coordination. The VR engine may supplement a patient's impairment profile, e.g., as a potential impairment, with one or more of those conditions.

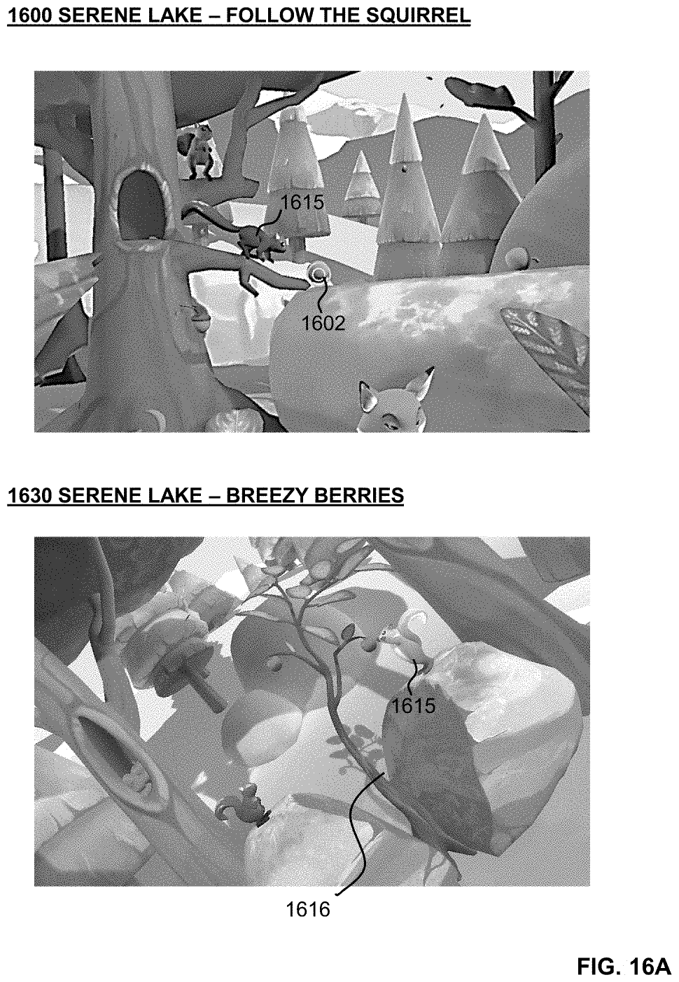

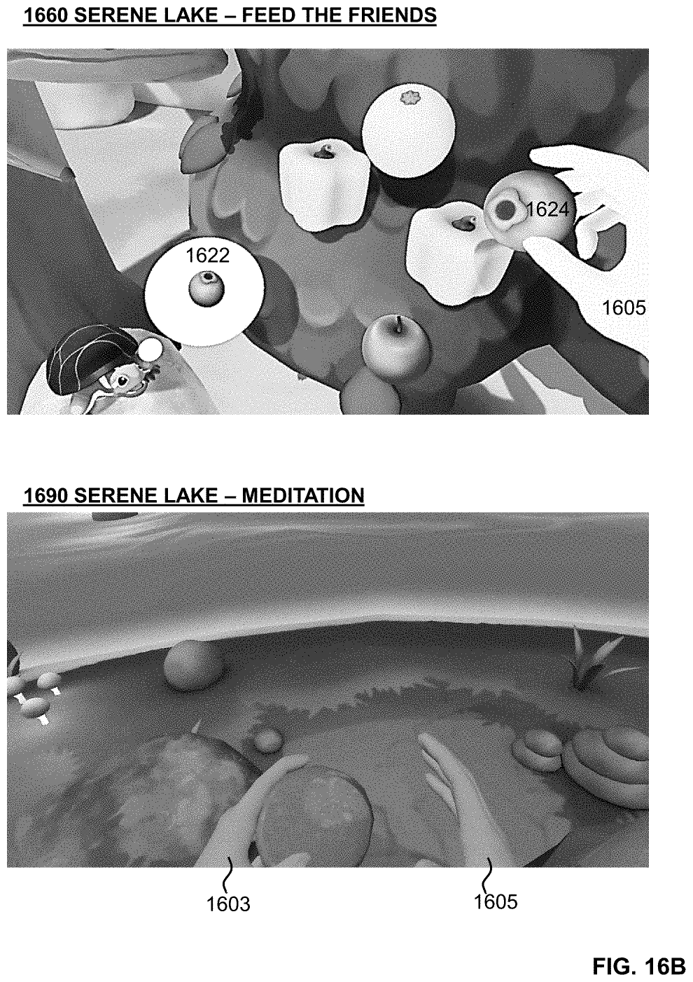

At step 624, the VR engine analyzes comprehension in activity performance data in view of impairment profile. For example, in activities like Feed the Friends of Serene Lake, depicted in FIG. 16B, the patient is asked to feed animals a requested berry from a bush of various berries in different colors, shapes, and sizes. A count in Feed the Friends of incorrect berries fed may indicate issues with comprehension. In the activity Stamp Stand of Mindful Market, depicted in FIG. 13B, groups of stamps are sold to customers requesting stamps totaling a specific dollar amount. A count in Stamp Stand of incorrect stamp amounts may indicate issues with comprehension.

At step 630, the VR engine determines if the activity performance data is below a predetermined threshold for comprehension. For example, in Feed the Friends, a count of incorrect berries greater than, e.g., 7, may indicate issues with comprehension. In the activity Stamp Stand, a count of, e.g., 5 or more incorrect stamp amounts by more or less than $5 may indicate issues with comprehension.

If the VR engine determines the activity performance is lower than the predetermined threshold for comprehension then, at step 632, the patient's impairment profile is supplemented. For example, in Feed the Friends, a count of incorrect berries greater than, e.g., 7, may indicate issues with regard to object recognition, color and shape matching, and/or sustained attention skills. In the activity Stamp Stand, a count of, e.g., 5 or more incorrect stamp amounts by more or less than $5 may indicate issues with working memory, matching, stimuli tolerance and/or sustained attention skills. The VR engine may supplement a patient's impairment profile, e.g., as a potential impairment, with one or more of those conditions.

After step 632, further activity performance data may be accessed at step 602, e.g., to restart process 600. Upon supplementing a profile with conditions (or prior to adding anything) a warning may be provided to a therapist or supervisor to check. For instance, a potential impairment listed as one or more of conditions may need urgent follow-up with a doctor.

Some embodiments may compare activity performance data with other thresholds, e.g., to identify other potential physical, neurological, cognitive, and/or sensory impairments and conditions. Some activities may directly or indirectly test for certain impairments or conditions. For instance, patients may be shown images to test for color blindness or provided sound tests to determine hearing levels. The VR engine may supplement a patient's impairment profile, e.g., as a potential impairment, with one or more of those conditions.

### An Illustrative Virtual Reality System to Treat Various Impairments

Disclosed herein is an illustrative medical device system including a virtual reality (VR) system to enable therapy for a patient. Such a VR medical device system may include a headset, sensors, a therapist tablet, among other hardware to enable exercises and activities to train (or re-train) a patient's body movements.

As described herein, VR systems capable for use in physical therapy may be tailored to be durable, portable and allow for quick and consistent setup. In some embodiments, a virtual reality system for therapy may be a modified commercial VR system using, e.g., a headset and several body sensors configured for wireless communication. A VR system capable of use for therapy may need to collect patient movement data. In some embodiments, sensors, placed on the patient's body, can translate patient body movement to the VR system for animation of a VR avatar. Sensor data may also be used to measure patient movement and determine motion for patient body parts.

FIG. 7A is a diagram of an illustrative system, in accordance with some embodiments of the disclosure. A VR system may include a clinician tablet 210, head-mounted display 201 (HMD or headset), small sensors 202, and large sensor 202B. Large sensor 202B may comprise transmitters, in some embodiments, and be referred to as wireless transmitter module 202B. Some embodiments may include sensor chargers, router, router battery, headset controller, power cords, USB cables, and other VR system equipment.

Clinician tablet 210 may be configured to use a touch screen, a power/lock button that turns the component on or off, and a charger/accessory port, e.g., USB-C. For instance, pressing the power button on clinician tablet 210 may power on the tablet or restart the tablet. Once clinician tablet 210 is powered on, a therapist or supervisor may access a user interface and be able to log in; add or select a patient; initialize and sync sensors; select, start, modify, or end a therapy session; view data; and/or log out.

Headset 201 may comprise a power button that turns the component on or off, as well as a charger/accessory port, e.g., USB-C. Headset 201 may also provide visual feedback of virtual reality applications in concert with the clinician tablet and the small and large sensors.

Charging headset 201 may be performed by plugging a headset power cord into the storage dock or an outlet. To turn on headset 201 or restart headset 201, the power button may be pressed. A power button may be on top of the headset. Some embodiments may include a headset controller used to access system settings. For instance, a headset controller may be used only in certain troubleshooting and administrative tasks and not necessarily during patient therapy. Buttons on the controller may be used to control power, connect to headset 201, access settings, or control volume.

The large sensor 202B and small sensors 202 are equipped with mechanical and electrical components that measure position and orientation in physical space and then translate that information to construct a virtual environment. Sensors 202 are turned off and charged when placed in the charging station. Sensors 202 turn on and attempt to sync when removed from the charging station. The sensor charger acts as a dock to store and charge the sensors. In some embodiments, sensors may be placed in sensor bands on a patient. Sensor bands 205, as depicted in FIGS. 7B-C, are typically required for use and are provided separately for each patient for hygienic purposes. In some embodiments, sensors may be miniaturized and may be placed, mounted, fastened, or pasted directly onto a user.

As shown in illustrative FIG. 7A, various systems disclosed herein consist of a set of position and orientation sensors that are worn by a VR participant, e.g., a therapy patient. These sensors communicate with HMD 201, which immerses the patient in a VR experience. An HMD suitable for VR often comprises one or more displays to enable stereoscopic three-dimensional (3D) images. Such internal displays are typically high-resolution (e.g., 2880×1600 or better) and offer high refresh rate (e.g., 75 Hz). The displays are configured to present 3D images to the patient. VR headsets typically include speakers and microphones for deeper immersion.

HMD 201 is a piece central to immersing a patient in a virtual world in terms of presentation and movement. A headset may allow, for instance, a wide field of view (e.g., 110°) and tracking along six degrees of freedom. HMD 201 may include cameras, accelerometers, gyroscopes, and proximity sensors. VR headsets typically include a processor, usually in the form of a system on a chip (SoC), and memory. In some embodiments, headsets may also use, for example, additional cameras as safety features to help users avoid real-world obstacles. HMD 201 may comprise more than one connectivity option in order to communicate with the therapist's tablet. For instance, an HMD 201 may use an SoC that features WiFi and Bluetooth connectivity, in addition to an available USB connection (e.g., USB Type-C). The USB-C connection may also be used to charge the built-in rechargeable battery for the headset.

A supervisor, such as a health care provider or therapist, may use a tablet, e.g., tablet 210 depicted in FIG. 7A, to control the patient's experience. In some embodiments, tablet 210 runs an application and communicates with a router to cloud software configured to authenticate users and store information. Tablet 210 may communicate with HMD 201 in order to initiate HMD applications, collect relayed sensor data, and update records on the cloud servers. Tablet 210 may be stored in the portable container and plugged in to charge, e.g., via a USB plug.

In some embodiments, such as depicted in FIGS. 7B-C, sensors 202 are placed on the body in particular places to measure body movement and relay the measurements for translation and animation of a VR avatar. Sensors 202 may be strapped to a body via bands 205. In some embodiments, each patient may have her own set of bands 205 to minimize hygiene issues.

A wireless transmitter module (WTM) 202B may be worn on a sensor band 205B that is laid over the patient's shoulders. WTM 202B sits between the patient's shoulder blades on their back. Wireless sensor modules 202 (e.g., sensors or WSMs) are worn just above each elbow, strapped to the back of each hand, and on a pelvis band that positions a sensor adjacent to the patient's sacrum on their back. In some embodiments, each WSM communicates its position and orientation in real-time with an HMD Accessory located on the HMD. Each sensor 202 may learn its relative position and orientation to the WTM, e.g., via calibration.

The HMD accessory may include a sensor 202A that may allow it to learn its position relative to WTM 202B, which then allows the HMD to know where in physical space all the WSMs and WTM are located. In some embodiments, each sensor 202 communicates independently with the HMD accessory which then transmits its data to HMD 201, e.g., via a USB-C connection. In some embodiments, each sensor 202 communicates its position and orientation in real-time with WTM 202B, which is in wireless communication with HMD 201. In some embodiments HMD 201 may be connected to input supplying other data such as biometric feedback data. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, respiratory monitors, light sensors, cameras, sensors, and other biometric devices. Biometric feedback, along with other performance data, can indicate more subtle changes to the patient's body or physiology as well as mental state, e.g., when a patient is stressed, comfortable, distracted, tired, over-worked, under-worked, over-stimulated, confused, overwhelmed, excited, engaged, disengaged, and more. In some embodiments, such devices measuring biometric feedback may be connected to the HMD and/or the supervisor tablet via USB, Bluetooth, Wi-Fi, radio frequency, and other mechanisms of networking and communication.

A VR environment rendering engine on HMD 201 (sometimes referred to herein as a “VR application”), such as the Unreal Engine™, uses the position and orientation data to create an avatar that mimics the patient's movement.

A patient or player may “become” their avatar when they log in to a virtual reality activity. When the player moves their body, they see their avatar move accordingly. Sensors in the headset may allow the patient to move the avatar's head, e.g., even before body sensors are placed on the patient. A system that achieves consistent high-quality tracking facilitates the patient's movements to be accurately mapped onto an avatar.

Sensors 202 may be placed on the body, e.g., of a patient by a therapist, in particular locations to sense and/or translate body movements. The system can use measurements of position and orientation of sensors placed in key places to determine movement of body parts in the real world and translate such movement to the virtual world. In some embodiments, a VR system may collect performance data for therapeutic analysis of a patient's movements and range of motion.

In some embodiments, systems and methods of the present disclosure may use electromagnetic tracking, optical tracking, infrared tracking, accelerometers, magnetometers, gyroscopes, myoelectric tracking, other tracking techniques, or a combination of one or more of such tracking methods. The tracking systems may be parts of a computing system as disclosed herein. The tracking tools may exist on one or more circuit boards within the VR system (see FIG. 9) where they may monitor one or more users to perform one or more functions such as capturing, analyzing, and/or tracking a subject's movement. In some cases, a VR system may utilize more than one tracking method to improve reliability, accuracy, and precision.

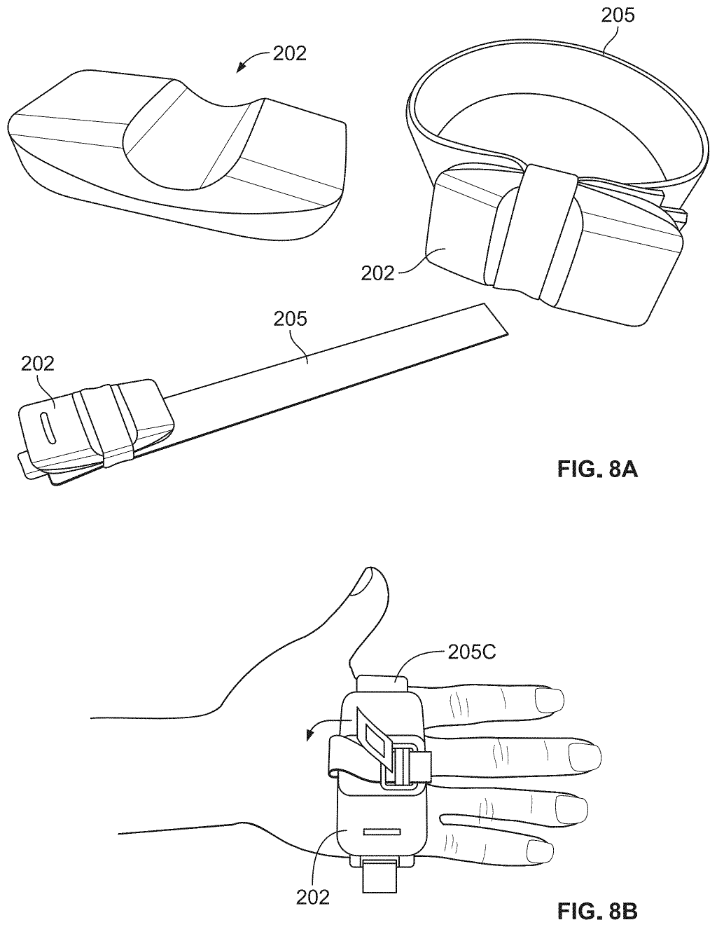

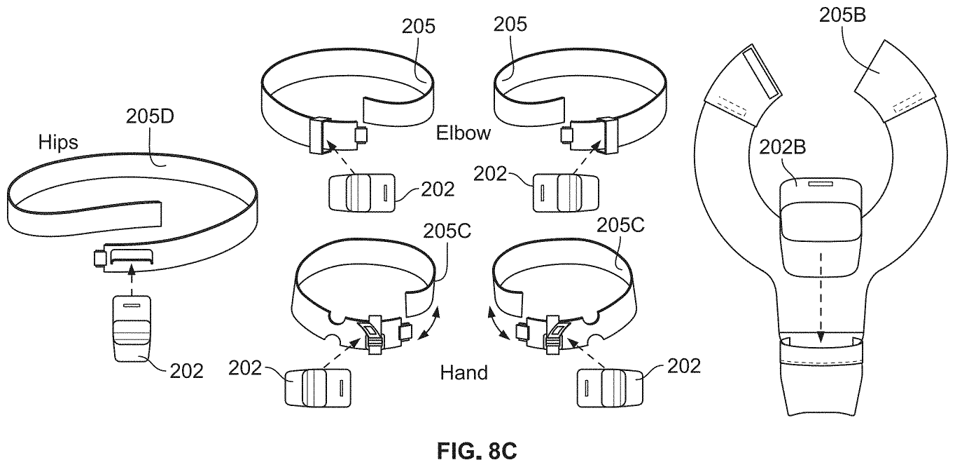

FIGS. 8A-C illustrate examples of wearable sensors 202 and bands 205. In some embodiments, bands 205 may include elastic loops to hold the sensors. In some embodiments, bands 205 may include additional loops, buckles and/or Velcro straps to hold the sensors. For instance, bands 205 for hands may require extra secureness as a patient's hands may be moved at a greater speed and could throw or project a sensor in the air if it is not securely fastened. FIG. 8B illustrates an exemplary embodiment with a slide buckle.

Sensors 202 may be attached to body parts via band 205. In some embodiments, a therapist attaches sensors 202 to proper areas of a patient's body. For example, a patient may not be physically able to attach band 205 to herself. In some embodiments, each patient may have her own set of bands 205 to minimize hygiene issues. In some embodiments, a therapist may bring a portable case to a patient's room or home for therapy. The sensors may include contact ports for charging each sensor's battery while storing and transporting in the container, such as the container depicted in FIG. 7A.

As illustrated in FIG. 8C, sensors 202 are placed in bands 205 prior to placement on a patient. In some embodiments, sensors 202 may be placed onto bands 205 by sliding them into the elasticized loops. The large sensor, WTM 202B, is placed into a pocket of shoulder band 205B. Sensors 202 may be placed above the elbows, on the back of the hands, and at the lower back (sacrum). In some embodiments, sensors may be used at the knees and/or ankles. Sensors 202 may be placed, e.g., by a therapist, on a patient while the patient is sitting on a bench (or chair) with his hands on his knees. Sensor band 205D to be used as a hip sensor 202 has a sufficient length to encircle a patient's waist.

Once sensors 202 are placed in bands 205, each band may be placed on a body part, e.g., according to FIG. 7C. In some embodiments, shoulder band 205B may require connection of a hook and loop fastener. An elbow band 205 holding a sensor 202 should sit behind the patient's elbow. In some embodiments, hand sensor bands 205C may have one or more buckles to, e.g., fasten sensors 202 more securely, as depicted in FIG. 8B.

Each of sensors 202 may be placed at any of the suitable locations, e.g., as depicted in FIG. 7C. After sensors 202 have been placed on the body, they may be assigned or calibrated for each corresponding body part.

Generally, sensor assignment may be based on the position of each sensor 202. Sometimes, such as cases where patients have varying height discrepancies, assigning a sensor merely based on height is not practical. In some embodiments, sensor assignment may be based on relative position to, e.g., wireless transmitter module 202B.

FIG. 9 depicts an illustrative arrangement for various elements of a system, e.g., an HMD and sensors of FIGS. 7A-D. The arrangement includes one or more printed circuit boards (PCBs). In general terms, the elements of this arrangement track, model, and display a visual representation of the participant (e.g., a patient avatar) in the VR world by running software including the aforementioned VR application of HMD 201.

The arrangement shown in FIG. 9 includes one or more sensors 902, processors 960, graphic processing units (GPUs) 920, video encoder/video codec 940, sound cards 946, transmitter modules 910, network interfaces 980, and light emitting diodes (LEDs) 969. These components may be housed on a local computing system or may be remote components in wired or wireless connection with a local computing system (e.g., a remote server, a cloud, a mobile device, a connected device, etc.). Connections between components may be facilitated by one or more buses, such as bus 914, bus 934, bus 948, bus 984, and bus 964 (e.g., peripheral component interconnects (PCI) bus, PCI-Express bus, or universal serial bus (USB)). With such buses, the computing environment may be capable of integrating numerous components, numerous PCBs, and/or numerous remote computing systems.

One or more system management controllers, such as system management controller 912 or system management controller 932, may provide data transmission management functions between the buses and the components they integrate. For instance, system management controller 912 provides data transmission management functions between bus 914 and sensors 902. System management controller 932 provides data transmission management functions between bus 934 and GPU 920. Such management controllers may facilitate the arrangements orchestration of these components that may each utilize separate instructions within defined time frames to execute applications. Network interface 980 may include an ethernet connection or a component that forms a wireless connection, e.g., 802.11b, g, a, or n connection (WiFi), to a local area network (LAN) 987, wide area network (WAN) 983, intranet 985, or internet 981. Network controller 982 provides data transmission management functions between bus 984 and network interface 980.

Processor(s) 960 and GPU 920 may execute a number of instructions, such as machine-readable instructions. The instructions may include instructions for receiving, storing, processing, and transmitting tracking data from various sources, such as electromagnetic (EM) sensors 903, optical sensors 904, infrared (IR) sensors 907, inertial measurement units (IMUs) sensors 905, and/or myoelectric sensors 906. The tracking data may be communicated to processor(s) 960 by either a wired or wireless communication link, e.g., transmitter 910. Upon receiving tracking data, processor(s) 960 may execute an instruction to permanently or temporarily store the tracking data in memory 962 such as, e.g., random access memory (RAM), read only memory (ROM), cache, flash memory, hard disk, or other suitable storage component. Memory may be a separate component, such as memory 968, in communication with processor(s) 960 or may be integrated into processor(s) 960, such as memory 962, as depicted.

Processor(s) 960 may also execute instructions for constructing an instance of virtual space. The instance may be hosted on an external server and may persist and undergo changes even when a participant is not logged in to said instance. In some embodiments, the instance may be participant-specific, and the data required to construct it may be stored locally. In such an embodiment, new instance data may be distributed as updates that users download from an external source into local memory. In some exemplary embodiments, the instance of virtual space may include a virtual volume of space, a virtual topography (e.g., ground, mountains, lakes), virtual objects, and virtual characters (e.g., non-player characters “NPCs”). The instance may be constructed and/or rendered in 2D or 3D. The rendering may offer the viewer a first-person or third-person perspective. A first-person perspective may include displaying the virtual world from the eyes of the avatar and allowing the patient to view body movements from the avatar's perspective. A third-person perspective may include displaying the virtual world from, for example, behind the avatar to allow someone to view body movements from a different perspective. The instance may include properties of physics, such as gravity, magnetism, mass, force, velocity, and acceleration, which cause the virtual objects in the virtual space to behave in a manner at least visually similar to the behaviors of real objects in real space.

Processor(s) 960 may execute a program (e.g., the Unreal Engine or VR applications discussed above) for analyzing and modeling tracking data. For instance, processor(s) 960 may execute a program that analyzes the tracking data it receives according to algorithms described above, along with other related pertinent mathematical formulas. Such a program may incorporate a graphics processing unit (GPU) 920 that is capable of translating tracking data into 3D models. GPU 920 may utilize shader engine 928, vertex animation 924, and linear blend skinning algorithms. In some instances, processor(s) 960 or a CPU may at least partially assist the GPU in making such calculations. This allows GPU 920 to dedicate more resources to the task of converting 3D scene data to the projected render buffer. GPU 920 may refine the 3D model by using one or more algorithms, such as an algorithm learned on biomechanical movements, a cascading algorithm that converges on a solution by parsing and incrementally considering several sources of tracking data, an inverse kinematics (IK) engine 930, a proportionality algorithm, and other algorithms related to data processing and animation techniques. After GPU 920 constructs a suitable 3D model, processor(s) 960 executes a program to transmit data for the 3D model to another component of the computing environment (or to a peripheral component in communication with the computing environment) that is capable of displaying the model, such as display 950.

In some embodiments, GPU 920 transfers the 3D model to a video encoder or a video codec 940 via a bus, which then transfers information representative of the 3D model to a suitable display 950. The 3D model may be representative of a virtual entity that can be displayed in an instance of virtual space, e.g., an avatar. The virtual entity is capable of interacting with the virtual topography, virtual objects, and virtual characters within virtual space. The virtual entity is controlled by a user's movements, as interpreted by sensors 902 communicating with the system. Display 950 may display a Patient View. The patient's real-world movements are reflected by the avatar in the virtual world. The virtual world may be viewed in the headset in 3D and monitored on the tablet in two dimensions. In some embodiments, the VR world is an activity that provides feedback and rewards based on the patient's ability to complete activities. Data from the in-world avatar is transmitted from the HMD to the tablet to the cloud, where it is stored for later analysis. An illustrative architectural diagram of such elements in accordance with some embodiments is depicted in FIG. 10.

A VR system may also comprise display 970, which is connected to the computing environment via transmitter 972. Display 970 may be a component of a clinician tablet. For instance, a supervisor or operator, such as a therapist, may securely log in to a clinician tablet, coupled to the system, to observe and direct the patient to participate in various activities and adjust the parameters of the activities to best suit the patient's ability level. Display 970 may depict a view of the avatar and/or replicate the view of the HMD.

In some embodiments, HMD 201 may be the same as or similar to HMD 1010 in FIG. 10. In some embodiments, HMD 1010 runs a version of Android that is provided by HTC (e.g., a headset manufacturer) and the VR application is an Unreal application, e.g., Unreal Application 1016, encoded in an Android package (.apk). The .apk comprises a set of custom plugins: WVR, WaveVR, SixenseCore, SixenseLib, and MVICore. The WVR and WaveVR plugins allow the Unreal application to communicate with the VR headset's functionality. The SixenseCore, SixenseLib, and MVICore plugins allow Unreal Application 1016 to communicate with the HMD accessory and sensors that communicate with the HMD via USB-C. The Unreal Application comprises code that records the position and orientation (P&O) data of the hardware sensors and translates that data into a patient avatar, which mimics the patient's motion within the VR world. An avatar can be used, for example, to infer and measure the patient's real-world range of motion. The Unreal application of the HMD includes an avatar solver as described, for example, below.

The clinician operator device, clinician tablet 1020, runs a native application (e.g., Android application 1025) that allows an operator such as a therapist to control a patient's experience. Cloud server 1050 includes a combination of software that manages authentication, data storage and retrieval, and hosts the user interface, which runs on the tablet. This can be accessed by tablet 1020. Tablet 1020 has several modules.

As depicted in FIG. 10, the first part of tablet software is a mobile device management (MDM) 1024 layer, configured to control what software runs on the tablet, enable/disable the software remotely, and remotely upgrade the tablet applications.

The second part is an application, e.g., Android Application 1025, configured to allow an operator to control the software of HMD 1010. In some embodiments, the application may be a native application. A native application, in turn, may comprise two parts, e.g., (1) socket host 1026 configured to receive native socket communications from the HMD and translate that content into web sockets, e.g., web sockets 1027, that a web browser can easily interpret; and (2) a web browser 1028, which is what the operator sees on the tablet screen. The web browser may receive data from the HMD via the socket host 1026, which translates the HMD's native socket communication 1018 into web sockets 1027, and it may receive UI/UX information from a file server 1052 in cloud 1050. Tablet 1020 comprises web browser 1028, which may incorporate a real-time 3D engine, such as Babylon.js, using a JavaScript library for displaying 3D graphics in web browser 1028 via HTML5. For instance, a real-time 3D engine, such as Babylon.js, may render 3D graphics, e.g., in web browser 1028 on clinician tablet 1020, based on received skeletal data from an avatar solver in the Unreal Engine 1016 stored and executed on HMD 1010. In some embodiments, rather than Android Application 1026, there may be a web application or other software to communicate with file server 1052 in cloud 1050. In some instances, an application of Tablet 1020 may use, e.g., Web Real-Time Communication (WebRTC) to facilitate peer-to-peer communication without plugins, native apps, and/or web sockets.

The cloud software, e.g., cloud 1050, has several different, interconnected parts configured to communicate with the tablet software: authorization and API server 1062, GraphQL server 1064, and file server (static web host) 1052.

In some embodiments, authorization and API server 1062 may be used as a gatekeeper. For example, when an operator attempts to log in to the system, the tablet communicates with the authorization server. This server ensures that interactions (e.g., queries, updates, etc.) are authorized based on session variables such as operator's role, the health care organization, and the current patient. This server, or group of servers, communicates with several parts of the system: (a) a key value store 1054, which is a clustered session cache that stores and allows quick retrieval of session variables; (b) a GraphQL server 1064, as discussed below, which is used to access the back-end database in order to populate the key value store, and also for some calls to the application programming interface (API); (c) an identity server 1056 for handling the user login process; and (d) a secrets manager 1058 for injecting service passwords (relational database, identity database, identity server, key value store) into the environment in lieu of hard coding.

When the tablet requests data, it will communicate with the GraphQL server 1064, which will, in turn, communicate with several parts: (1) the authorization and API server 1062; (2) the secrets manager 1058, and (3) a relational database 1053 storing data for the system. Data stored by the relational database 1053 may include, for instance, profile data, session data, application data, activity performance data, and motion data.

In some embodiments, profile data may include information used to identify the patient, such as a name or an alias. Session data may comprise information about the patient's previous sessions, as well as, for example, a “free text” field into which the therapist can input unrestricted text, and a log 1055 of the patient's previous activity. Logs 1055 are typically used for session data and may include, for example, total activity time, e.g., how long the patient was actively engaged with individual activities; activity summary, e.g., a list of which activities the patient performed, and how long they engaged with each on; and settings and results for each activity. Activity performance data may incorporate information about the patient's progression through the activity content of the VR world. Motion data may include specific range-of-motion (ROM) data that may be saved about the patient's movement over the course of each activity and session, so that therapists can compare session data to previous sessions' data.

In some embodiments, file server 1052 may serve the tablet software's website as a static web host.

### Exemplary VR Applications and Activities to Treat Various Impairments

In some embodiments, the activities and exercises may include gazing activities that require the player to turn and look. A gaze activity may be presented as a hide-and-seek activity, a follow-and-seek exercise, or a gaze and trigger activity. The activities may include sun rising activities that require the player to raise his or her arms. The activities may include hot air balloon exercise s that require the player to lean and bend. The activities may include bird placing activities that require the player to reach and place. The exercises may include a soccer-like activity that requires a player to block and/or dodge projectiles. These activities may be presented as sandbox activities, with no clear win condition or end point. Some of these may be free-play environments presented as an endless interactive lobby. Sandbox versions of the activities may be typically used to introduce the player to the activity mechanics, and it allows them to explore the specific exercise's unique perspective of the virtual reality environment. Additionally, the sandbox activities may allow a therapist to use objects to augment and customize therapy, such as with resistance bands, weights, and the like. After the player has learned how the exercise mechanics works, they can be loaded into a version of the activity with a clear objective. In these versions of the activity, the player's movements may be tracked and recorded. After completing the prescribed number of repetitions (reps) of the therapeutic exercise (a number that is adjustable), the activity may come to an end and the player may be rewarded for completing it. In some embodiments, activities and exercises may be dynamically adjusted during the activity to optimize patient engagement and/or therapeutic benefits.

The transition from activity to activity may be seamless. Several transition options may be employed. The screen may simply fade to black, and slowly reload through a fade from black. A score board or a preview of the next exercise may be used to distract the player during transition. A slow and progressive transition ensures that the patient is not startled by a sudden change of their entire visual environment. This slow progression may limit any disorientation that might occurs from a total, quick change in scenery while in VR.

At the end of an activity or exercise session, the player may be granted a particular view of the VR environment, such as a birds-eye view of the world or area. From this height, players may be offered a view of an ever-changing village. Such changes in the village are a direct response to the player's exercise progression, and therefore offer a visual indication of progression. These changes will continue as the player progresses through the activities to provide long-term feedback visual cues. Likewise, such views of the village may provide the best visual indicia of progress for sharing with family members or on social media. Positive feedback from family and friends is especially important when rehab progress is limited. These images will help illustrate how hard the player has been working and they will provide an objective measure of progress when, perhaps, physically the player feels little, if any, progress. Such features may enhance the positivity of the therapy experience and helps fulfill the VR activities' overall goals to be as positive as possible while to encouraging continued participation and enthusiasm.

FIG. 11A depicts illustrative user interfaces for a VR therapy activity, Music in Motion, in accordance with some embodiments of the disclosure. For instance, some embodiments may include an activity or activity called “Music in Motion.” Music in Motion may be focused to help patients through rehabilitation therapy and increase their range of motion using rhythm-based activities. Some embodiments may include activities within its VR world e.g., “Song Safari,” “Lean into the Music,” “Reach for the Rhythm,” and “Twist with the Tempo.” As an overarching goal, some embodiments may include a summary at the end of a session including data from the combined activities, e.g., rewards, scores, times, etc. Some cases may also include a pause functionality during play time. Some cases may have imagery of arms to complete the activity.

In Song Safari 1100, a patient may be guided through the activity of picking and or finding creatures, e.g., as a seek-and-find. The user may control their cursor by moving a VR reticle 1102 utilizing cervical range of motion to complete the task. In some embodiments, as a patient's success is registered, their meter may be filled. The user may need to look around the world in some embodiments to find the creatures that are in the world, e.g., hidden bunny 1112. In some embodiments, to succeed in the exercise, a patient may need to find a specific number of creatures which may be used to fill a success meter 1110. In some cases, the activity may adjust based on the user's activity or by their therapist's request which may alter the activity's difficulty, e.g., changing the number of creatures present at a time, how many creatures need to be found to complete a level, possible time limits, how long the reticle (e.g., cursor, gaze pointer) needs to be on the target to register a success, and the speed at which the target moves. Some embodiments may include a timer feature 1104, environmental attributes, in-activity counters 1108 and/or score cards 100, and/or visual/auditory features to denote the success or failure of a patient, there may also be some functionality to disable this portion of the activity.

In Lean into the Music 1130, the goal may be for the user to utilize trunk control to complete the activities. In some embodiments, a patient's objective may be to feed an in-activity creature, e.g., a canary at cursor 1102. Some cases may feed the creature by having a patient lean to incite the creature's movement. The candy may come in from specific locations in the activity, e.g., heading toward the user in a stream at the center of the screen. In some embodiments, the goal may be for the user to feed the creature the candies consecutively in order to get increased points. As a patient progresses through the exercise, they may face increased challenges, e.g., greater variation in the candies' locations, different point values based on candy type, external stimuli introduced for distraction, some candies to avoid, etc. There may be options for the user in some embodiments to choose what music they wish to listen to. Some cases may let the user repeat the level, bask in their rewards, or pick a new activity.

FIG. 11B depicts further illustrative user interfaces for a VR therapy activity, Music in Motion, in accordance with some embodiments of the disclosure. Reach for the Rhythm 1160 may focus on engaging a user's functional reach. A patient may have a goal to reach for pieces of candy as they come toward them. The candies may instruct the user regarding which hand they should use to collect the groups of candy in some embodiments, e.g., using differing colors. The activity may vary by song choices or difficulty, e.g., including difficult to reach positions, overlap between different candy colors, varying speeds, candies that require both hands, external distractions, candies to avoid, etc. Some embodiments may include rewards to indicate success or notifications of failures. There may be an additional “Excite Meter,” e.g., as part of success meter 1110, which can also indicate success by character interaction, fireworks, lights, sounds, etc. In some embodiments, buttons may be placed within the activity user interface, such as pause button 1114, which a user may virtually press to pause the activity.

Twist with the Tempo 1180 may be included in some embodiments. Twist with the Tempo may work with a patient's functional reach and their wrist rotation (e.g., pronation or supination). Some embodiments may direct where a patient should place their hand 1103. The activity may have objects 1182 that the player is aiming for, e.g., ice cream cones, tilted for a patient to replicate in their movement. For instance, object 1182 is titled different than object 1184, and user my tilt outline 1183 to match up with each object as it approaches. Some cases may include notifications of success for individual objects that are caught by the user, but there may be other general indicators, e.g., more creatures coming out to play, objects in the environment dancing, fireworks in the sky, etc. The activity may vary by song choices or difficulty, e.g., including difficult to reach positions, overlap between different object colors, varying speeds, objects that require both hands, external distractions, objects to avoid, specific hand positions, frequency and distance of the objects around a patient may vary, etc. During some embodiments, the user might also need to use both hands to reach and/or grab all incoming objects.

FIG. 12A depicts illustrative user interfaces for a VR therapy activity, Pleasant Cove, in accordance with some embodiments of the disclosure. Some embodiments may include an activity or activity called “Pleasant Cove.” Pleasant Cove activities may be used, for instance, to improve comfort, confidence, and engagement. Pleasant Cove may present low-intensity tasks and activities to exercise and improve memory of a subject. Generally, Pleasant Cove activities have the goal of engaging the subject and immersing her in a calm VR world.

Some embodiments may feature at least four activities in the Pleasant Cove virtual world, e.g., “Bountiful Birdseed,” “Playful Percussion,” “Green Thumb Gardening,” and “ADL Cards.” Each of these exercises has the goal to develop patient comfort, confidence, and engagement through relaxed, low-intensity memory-inspired activities. A subject may select an activity in Pleasant Cove by opting-in to a task, for example, when looking at a specific spot and accepting (or declining) a prompt. In some embodiments, a supervisor (e.g., therapist) may select a specific activity.

In Bountiful Birdseed 1200, a user may interact with a virtual bird 1210 named “Shy Bird.” For instance, visual cues may be provided to shake food 1206 on certain areas of a floor or table to coax the bird to come closer. In some cases, in-activity visual cues may identify that, e.g., after feeding Shy Bird one or more times and the bird is close enough to the virtual user, that the birdseed 1206 may be shaken into the subject's open virtual hand 1203 so that the bird will land on the hand and eat food 1206A from the palm. In some embodiments, when the task is complete, a celebratory noise will be played and/or virtual confetti will appear to rain down. In some embodiments, one or more other birds flying around the Pleasant Cove environment may come and feed on the birdseed laid out on the floor, on a table, and or in a hand. Bountiful Birdseed is a relatively simple activity designed to promote comfortability in a VR world and encourage further engagement in Pleasant Cove and VR therapy.

Bountiful Birdseed 1200 may be used for subjects experiencing cognitive impairment or decline with symptoms of impaired attention, memory, psychomotor skills, and/or sequencing. In some embodiments, Bountiful Birdseed activities may be suitable for use with elder patients or patients experiencing forms of dementia.

Playful Percussion 1230 may be another task in Pleasant Cove. Playful Percussion allows a subject to play a VR xylophone-type instrument, e.g., xylophone 1104, by banging virtual mallets 1203A and 1205A on different keys. Some embodiments may use one of or both of left hand 1203 and right hand 1205. A subject may select a song to play from several available, familiar songs in a VR digital songbook 1238. For instance, a subject may select songs such as “Happy Birthday,” “Ode to Joy,” “Pop Goes the Weasel,” “Mary Had a Little Lamb,” “Jingle Bells,” or “Twinkle, Twinkle Little Star.” In some embodiments, a visual cue, such as an arrow 1236 or lighted-up key 1230, may be presented to the subject to indicate which key to hit next in sequence to produce the notes of the song. Playful Percussion is focused on sequencing and working memory while also engaging in psychomotor skills to follow the arrow to play the correct note for a song. In some embodiments, assistive features and modifications within this activity may include music volume and mute, visual cue adjustments, left- or right-handed dominance, as well as a one-handed mode. Some embodiments may include a “free play” mode. In some embodiments, bird 1210 may provide guidance.

FIG. 12B depicts further illustrative user interfaces for a VR therapy activity, Pleasant Cove, in accordance with some embodiments of the disclosure. Green Thumb Gardening 1260 is another activity in Pleasant Cove designed to produce comfort and engagement. Some embodiments may use one of or both of left hand 1203 and right hand 1205. In some embodiments, this activity may be used for subjects to practice their sequencing skills while peacefully arranging and caring for flowers 1272, 1274, et al. A subject may plant flowers and let them grow before arranging them. Some embodiments may have multiple modes where the subject can freely plant and arrange flowers, or they can play in a guided setting. In the free-play mode, the subject may be guided through the process of planting and arranging flowers e.g., shoveling the dirt in pot 1276, pouring seed types, watering the planted seeds, picking the flowers 1274 and 1272, placing them into an arrangement in foam 1266. In the guided setting, the goal may be to match the placement of flowers to an instruction card 1268 attached to the floral foam. In some embodiments, a visual cue, such as an arrow or dot 1262, may be presented to the subject to indicate where to place the flower that the subject has picked up in order to match the instructions. In some embodiments, therapists may collect performance data e.g., the amount of time in the activity along with data related to seeds planted, flowers grown, flowers picked, and the accuracy of the flower placement to the card. There may be a guide for a patient e.g., a bird to indicate the process that the user should follow to achieve the task. Bird 1201, for instance, may use carrier 1265 to fly the correctly placed flowers away upon completion. In some embodiments, specific flowers may be colored, e.g., yellow, to promote calmness and serenity during the task. Some embodiments may include varying levels of distractions, e.g., many flowers, many birds, many clouds, many sounds, etc.

ADL Cards 1280 is another activity in Pleasant Cove may be used to improve sequencing and impaired procedural memory by card matching, sequencing, or identification inspired by a real-life therapeutic technique. In some embodiments, the user may pick up a deck of cards, e.g., using one of or both of left hand 1203 and right hand 1205, from one position in front of them before placing the deck in another location. This deck may include ADL cards that are meant to help the subject with activities of daily living (ADLs) e.g., bathing, grooming, eating, dressing, and more. These ADLs may be images captured on the cards intended for users to practice sequencing and exercise impaired procedural memory by lining up the cards in the accurate order. For instance, card 182 indicates a procedure for showering is being tested. Blanks 1284 and 1286 are intended form cards 1292 and 1290 in the proper order, before card 1288 (rinse). Card 1292 (lather soap) should be placed in spot 1284, and card 1290 (wash) should be placed in spot 1286. In some embodiments, bird 1201 may provide some guidance and/or indication of correct placement. In some embodiments, data will be collected, e.g., capture the user's time spent in the activity, sequencing and sorting abilities, level reached while sequencing, and number of completed sequences. In some embodiments, there might be a focus on subjects with recoverable impaired cognition. There may be a visual symbol or action to indicate success or failure in the activity, e.g., a bird putting on a hat at the successful ordering of the cards.

FIG. 13A depicts illustrative user interfaces for a VR therapy activity, Mindful Market, in accordance with some embodiments of the disclosure. Some embodiments may include an activity or activity called “Mindful Market.” Mindful Market may be used, for instance, to improve comfort, confidence, and engagement. Mindful Market may be a cognitive-focused application designed to address impairments e.g., executive functioning, short-term and working memory, sequencing, stimuli tolerance and endurance, and resilience within the context of ADLs to reinforce functionality. Generally, Mindful Market activities have the goal of engaging the subject and immersing her in a calm VR world.

Some Embodiments of Mindful Market may include activities “Sandwich Shop,” “Harvest Helper,” and “Stamp Stand.” These activities may help in the context of activities of daily living (ADLs) in a safe environment. Embodiments may have a patient serve as a volunteer to help the community in the VR world through the activity's exercises. Different embodiments may include varying levels of audio stimulation, visual/environmental stimulation, non-playable character (NPC) reactions, interactable spaces within visible reach to minimize head movement. Patients may go through the activities at different paces due to their impairments.

In some embodiments of Mindful Market, there may be a Lobby Area where a patient waits. A patient and/or therapist may wait in a lobby to ensure proper setup and/or choose an activity. This Lobby area may be engaging auditorily and visually, peaceful, comfortable, and soothing while a patient is virtually there.

Sandwich Shop 1300 may be an activity in embodiments of Mindful Market. In Sandwich Shop 1300, a patient may watch from the perspective of a food vendor as a customer comes to their counter. Upon the customer's arrival, there may be a food order presented on the screen. There then may be the ingredients presented on the screen for a patient to fulfill these orders. Some embodiments may use one of or both of left hand 1303 and right hand 1305. A patient may need to pick up the items in their view to fulfill the order. For instance, a customer may request a sandwich 1306 with ingredients of white bread, bologna, and onions, and the user would have to prepare the correct sandwich 1312. If a patient chooses a wrong ingredient, they may need to dispose of the sandwich and start over in order to create the correct recipe for their customer. A patient may be able to look around the booth to see several features and different items that they can interact with from ingredient area 1310, as well as see customer reactions and the requested sandwich ingredients. There may be condiments and different foods to put on the sandwiches. Some embodiments may let the user handle the ingredients in multiple ways. Some embodiments may use both hands to function, e.g., grabbing bread from the left and grabbing condiments from the right. Having completed the sandwich, the patient may give the customer their completed sandwich for review and approval. The process of activity approval may include an indication of a patient's success, e.g., the customer smiling and taking pictures with the food.

Harvest Helper 1330 may be one of the activities included in Mindful Market's world. This activity may be useful to improve stimuli tolerance, working memory, and sorting abilities while also exercising shoulder flexion and trunk control. Some embodiments may have a patient help a character in the activity who is tossing them packages to the patient. Some embodiments may require both left hand 1303 and right hand 1305 together or separately. The activity may use visual aids to help instruct the user's movement, e.g., the silhouette of two hands 1303A and 1305B where they can catch the item being tossed to them and a highlighted position where the item should be placed. As the user is tossed the item 1340 to catch, they may receive the item and carefully place it in a specified location to complete the task. As the activity progresses, the user may catch items of different sizes or organize the different items by placing them in different groups, e.g., pumpkins in one position in the exercise and corn on the cob in another. Some embodiments of the activity may include an environmental or character reaction based on a patient's success or failure completing the activity. Some embodiments may dynamically adjust the object throw rate, object throw height, object size, object placement organization, etc. Some embodiments may dynamically adjust the accuracy needed to actually catch the object, lending help when the patient is struggling or stressed, as well as requiring more precise hand placement when the patient is having success.

FIG. 13B depicts further illustrative user interfaces for a VR therapy activity, Mindful Market, in accordance with some embodiments of the disclosure. Stamp Stand 1360 is another example activity within Mindful Market. Stamp Stand has potential uses working on mental math, working memory, cognitive ability, executive functioning, and the ADL of interpersonal transactions. In Stamp Stand 1330, a patient may look around the booth where they are working, e.g., to sell stamps of various amounts fitting a customer's request. In some embodiments, a customer will arrive to the stand with an approximate price for what they want to purchase and the items they want to receive. A patient's goal may then be to sell the customer a group of items (stamps) totaling close to the price that the customer wishes to pay. The patient may then look at their inventory to decide what to sell the customer. A patient may choose which items to sell the customer and take them from their current position in order to sell them. Some embodiments may use one of or both of left hand 1303 and right hand 1305. Some embodiments may include in-activity calculation features or may require a patient to complete mental math to come up with the total price for what they are giving to the customer. The patient may then hand the customer their purchase. Some embodiments may include encouraging environment or character reactions based on the user's success. Some embodiments may have varying levels of difficulty based upon the user's success, e.g., limiting the use of the calculator, having different value items, the customer requesting different items and/or amounts, more precise price requests, time limits for the transactions, and requirements to add the prices of differently valued items (e.g., both $0.35 stamps and $0.50 stamps). Some embodiments might have one or more of such settings adjusted dynamically during a user's session.

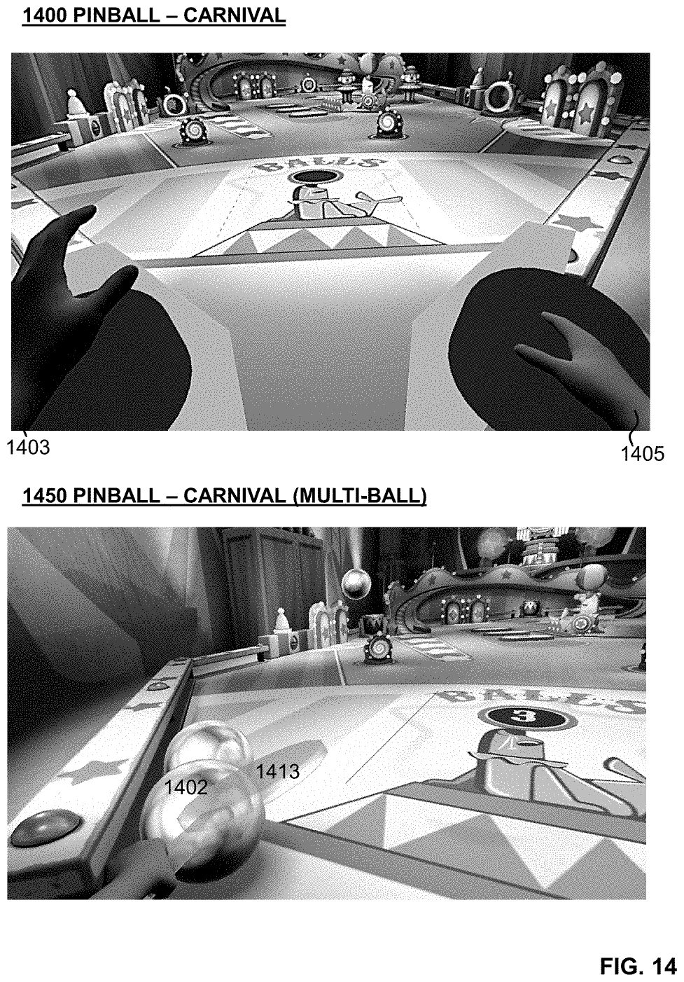

FIG. 14 depicts illustrative user interfaces for a VR therapy activity, Pinball, in accordance with some embodiments of the disclosure. Some embodiments may include an activity or activity called “Pinball.” Pinball 1400 may be used, for instance, to improve comfort, confidence, and engagement while working on a patient's range of motion to help with movement disorders. It may also help with coordination and timing as obstacles appear and disappear from the screen. Generally, Pinball 1400 activities have the goal of engaging the subject and immersing her in an engaging VR world. Some embodiments may use one of or both of left hand 1403 and right hand 1405.

Pinball 1400 and 1450 may instruct the user to come into contact with a ball 1402 using an object, e.g., typical pinball paddles at the end of the board or ping pong paddles 1413. In some embodiments, the user may interact with the ball(s) using paddles by “pressing” the buttons in the activity with a directed force. The goal of some embodiments may be to hit different locations within the activity's environment for points which may add up toward the user's score. Embodiments may include different sounds, animations and other effects to indicate their success in the activity. There may be limitations on the number of play attempts a user may have in the activity. Some embodiments may include more balls being introduced, e.g., pinball 1450, various levels of distractions, or increased requirements for precision in order to succeed. The environment may denote the number of balls, e.g., visually as a scoreboard on the floor of the activity. Some embodiments may indicate where the ball is despite visual obstructions, as well. In some embodiments, if all three balls are lost, the ball count is reduced.

Some embodiments may feature different pinball environments, themes, etc. with, e.g., different goals. For instance, in the activity “Alien Arrival” of Pinball, there may be a similar general goal of hitting the balls, while there are specific targets of “aliens” moving closer to the patient for them to knock down. Additionally, there may be the introduction of different interactions with the environment, e.g., Alien Arrival's elastic/spring border, and moving obstruction.

FIG. 15A depicts illustrative user interfaces for a VR therapy activity, Island Antics, in accordance with some embodiments of the disclosure. Some embodiments may include an activity or activity called “Island Antics.” Island Antics may be used, for instance, to help with motor and cognitive exercises for patients diagnosed with decreased ranges of motion, while also providing comfort, confidence, and engagement for their patients. Generally, Island Antics activities have the goal of engaging the subject and immersing her in an intriguing VR world.

Some embodiments of Island Antics may include multiple activities within its VR world including “Seagull Rescue,” “Citizen Crossing,” “Leaks and Lovers,” and “Coconut Chuck.” Each of these activities or exercises aims to comfort a patient while helping them acclimate to the tasks that they will encounter in their daily lives like the need for an increased range of motion. In some embodiments of these activities, the mode of play, free or guided, may be available for a patient's therapist to determine the best use. Embodiments of these activities may collect performance data, e.g., precision markers, time to complete task, number of tasks completed in a specified amount of time, comparisons of muscle dominance, etc. Some embodiments may use one of or both of left hand 1503 and right hand 1505.

In Seagull Rescue 1500, a patient may be given the opportunity to save seagulls. In some embodiments, these seagulls may be in danger of UFOs. Some embodiments require the user to save the seagulls from being taken by the UFOs by grabbing the vehicles 1502 in the process of abducting the seagulls and disposing of them, e.g., by throwing. Some embodiments may use the activities or motions in the activity to exercise trunk control, functional reach, and/or cross-body motions. This disposal process may take a similar form to the user throwing like a flying disk or Frisbee®. Some embodiments may use different signals, e.g., auditory or visual to encourage the user for their successful save of the seagulls. There may also be visual instructions in some embodiments, e.g., to tell the user how to dispose of the UFOs. Some embodiments may animate guidance path 1512 to instruct a preferred motion. Some embodiments may allow the user to look around the VR world that they are immersed in to see more of their surroundings and/or to determine where the UFOs are arriving from. In some embodiments, as a patient plays the activity, there may be highlighting to determine what UFO is being picked or thrown etc. As the players defeat the UFOs, some embodiments may require an allotted amount of time to protect the seagulls, a loss of all seagulls, a number of defeated vehicles, etc. In some embodiments, as the player progresses, the exercise's intensity may increase, e.g., the number of ships attacking the seagulls, the speed of the ships, the locations where the seagulls may be picked up, and/or where the ships are arriving from. Some cases may reward a patient by sending the ship away after success or have other outcomes to indicate a loss by the user.

In Citizen Crossing 1530, some embodiments may have the user help characters in the activity across broken paths. The purpose of this activity may be to help the users increase their range of motion, which may sometimes be enacted by holding characters with their hands 1505 and arms in order to save the citizens. In some cases, there may be instructional imagery to indicate the path for the user to take the activity's characters. Some embodiments may include progressions of the paths 1512 that the characters may take increasing and indicating where the citizens can or want to go or increasing where they can come from. Additionally, some cases may include indicators of the users' success, e.g., audio or visual cues that the player has accomplished their task. A patient may look around their surroundings to varying extents in some cases. There may also be a waving functionality in some embodiments. Some embodiments may include dynamic distractions such as air being blown to move characters less predictably.

FIG. 15B depicts further illustrative user interfaces for a VR therapy activity, Island Antics, in accordance with some embodiments of the disclosure. For instance, Leaks and Levers 1560 may occur in some embodiments of “Island Antics.” Leaks and Levers may be used to enable different movement patterns to help increase patients' ranges of motion. In some embodiments, the users may fix and turn back on valves through physical motion, e.g., flexion and extension of the patient's shoulder(s). Some embodiments may include instructional material, e.g., visual arrow paths 1512, to indicate how a patient should move in order to achieve the task. As a patient succeeds, in some embodiments, the tasks may introduce different patterns of movement, changes in the size and shape of the valve options, the movement required, require simultaneous movement, and/or bilateral movement to shut off the valves. Some cases may include indications of success through in-world activity queues, e.g., audio or visual acknowledgements of a patient's success or failure to achieve a task.

In Coconut Chuck 1580, a patient may have the goal to increase their range of motion. Some embodiments of this activity may include tasks where the goal is for the user to take the handle of the coconut slingshot, pull it a predetermined distance and then they may or may not need to fulfill a specific action to release the slingshot. In some embodiments, the user may aim for specific targets, or may aim freely. In some embodiments, increased precision may be required. In some cases, there may also be notification to the user that they have succeeded or failed in their task e.g., auditorily or visually.

FIG. 16A and FIG. 16B each depict illustrative user interfaces for a VR therapy activity, Serene Lake, in accordance with some embodiments of the disclosure. Some embodiments may include an activity or activity called “Serene Lake.” Serene Lake may be a low-stimuli environment which may help improve tolerance to sensory processing and visual/auditory tolerance such as those stemming from Traumatic Brain Injuries (TBI's), while also providing comfort, confidence, and engagement for their patients. Some embodiments may have applications may be for those needing to restore visual-spatial manipulation, color and shape matching, short term memory/recall, and sustained attention skills. Generally, Island Antics activities have the goal of engaging the subject and immersing her in an intriguing VR world. Some embodiments may use one of or both of left hand 1603 and right hand 1605.

Some embodiments of Serene Lake may include multiple sub-activities e.g., “Follow the Squirrel,” “Breezy Berries,” “Feed the Friends,” “Beaver Builders,” “Helping Hands,” “Target Match,” “Find the Pairs,” “Shell Game,” and “Meditation.” Some embodiments of these activities may have one or more non-playable characters who may join a patient throughout several activities along their journey to help them with the activities and provide comfort throughout the experience, e.g., a fox guide. There may be an intro hub, e.g., “The Glade,” where a therapist or patient may tweak comfort settings for an environment, e.g., light intensity, sound volume, environmental complexity (where extraneous creatures and objects may be removed), etc., based on the impairments of the patient which may remain for the activity's entirety.

Follow the Squirrel 1600 may track the user's gaze with cursor 1602 in a seek-and-find-style exercise. In some embodiments, the activity may direct a patient to use their head to follow the squirrel's movements as the squirrel 1615 finds acorns. Some embodiments may have the squirrel move in various directions along the tree as it is tracked. Some embodiments may switch, e.g., to require a patient to follow an acorn until it is picked up and then the squirrel as it is recovered. Some embodiments may have the creature, e.g., the squirrel, come slowly to a halt when the user's attention or eye contact strays from the creature. In some embodiments, the activity will end once all of the objects, e.g., the acorns, have been collected by the creature while others may have deeper activities and exercises by, e.g., including multiple levels of creatures. The activity may vary by intensity e.g., by increasing the number of creatures, needing to avoid certain objects, adding jumping capabilities, time limits, point systems, etc.

Breezy Berries 1630 may be incorporated in some embodiments. In some cases, a patient may control the activity by actively tilting their head and/or upper body to help bend a tree's trunk and/or branches to help a creature, e.g., a squirrel, reach the fruits on the trees, other body movements could also be used to activate the tree's movement. Some embodiments of the activity may include progress bars to indicate the amounts of fruits that have been reached by each creature. Some embodiments may introduce greater difficulty as the activity progresses, e.g., requiring movements may be required of different body parts, e.g., tilting the patient's head to move the tree trunk and/or a hand to move a specific branch of the plant, incorporating time limits, having the patient work with or against the wind, specifying specific fruits for specific creatures, or requiring some other sorting method.

In Feed the Friends 1660, a patient may see several creatures asking for specific items or foods. In some embodiments, a user may practice object recognition and command response to give the creature(s) what they are asking for, e.g., from a specific position in the VR activity. Some cases may have the animal take the item that they are asking for from the user when the user carries the object to overlap their position. For instance, a turtle may ask for blueberry 1622 and a patient may grab blueberry 1624, which matches blueberry 1622, with right hand 1605 and deliver it to the turtle to eat. Some embodiments may alter the difficulty of the activity by adjusting the settings, e.g., what creatures ask for fruit, how many can ask at once, the number of items available at a time, the variety of the fruits, the objects themselves to be fruits or berries depending on the impediment the exercise should be addressing (e.g., shape, pattern, or color matching), the amount of time that the creature may ask for the fruit, regrowth time, etc. In some embodiments, the gaze may be toggled on an off to let the user use their arms or their eyes to choose the fruits to feed the animals. The fruits may be animated to indicate that they are the wrong choice for the animal's chosen food.

Beaver Builders may place patients next to a gentle and calming waterfall to help creatures, e.g., a family of beavers, build something, e.g., a beaver dam. This activity may help patients through focus on clear object recognition and visuo-spatial manipulation. In some embodiments, the user's gaze reticle may be used to choose between multiple shapes to fill empty spots in the beaver's dam. The difficulty of shapes may be toggled in different embodiments to alter difficulty levels. Some cases may also introduce different obstacles as the user plays through an exercise, e.g., requirements to use several shapes to fill the holes, time limits, distractions, or size varying capabilities for the user.

Find the Pairs activity may require pattern recognition and memory effort for a user's success. Some embodiments may introduce a character, e.g., Kingfisher, to instruct a patient on how to complete the activities required of the activity. Some instances of this activity may require users to flip over cards to match pairs. This activity may be completed using a patient's hands (engaging cervical ROM) or gaze as chosen in the settings.

The Shell Game in some embodiments may work on a patient's memory and tracking skills. Some embodiments may use creatures, e.g., turtles, to hide a symbol. The activity may begin by showing a patient a specific creature that holds the symbol, then re-hide the symbol and shuffle the creatures before asking the patient to identify which of the creatures has the symbol. The level of difficulty may be toggles by a therapist in different embodiments, e.g., by determining the speed of the creatures' shuffling, the number of creatures being shuffled, the number of creatures in need of identification, etc. Some embodiments may also allow for a toggling of the visual stimuli depending on the user's impediments.

In Meditation 1690, a patient may relax or take a break if frustrated or overstimulated in the activity. This may be accessible at different points in different embodiments. In some cases, there may be a feature working with the patient's range of motion by skipping stones in the virtual world. This activity may let the stones fall into the water or skip across its surface depending upon the patient's movement in placing and/or throwing the virtual rocks.

Helping Hands may help a patient feel more comfortable by working with pattern matching, command response, and the patient's functional reach capacities. In some embodiments, a patient may be given several objects, e.g., cairn stones, to match with a tower made by a creature in the exercise, e.g., a baby beaver. Some cases may have the creature indicate where to place the object that matches the tower and its proper orientation. The activity's difficulty can be increased in some embodiments, as a patient works through the activities, e.g., adjusting the number of stones in the tower, the object distance, the activity repetitions, the creature's specificity of instructions, the time allowed for the user to complete the activity, the number and type of objects to choose from, etc.

Target Match as an activity may have the user fins creatures with symbols matching what the instructions or instructor, e.g., the Kingfisher, are asking for and may help a patient's working memory. A patient, in some embodiments, may need to remember previous symbols that they have found in order to find the creature with the intended symbol. Some cases may identify successful completion of the activity by selecting the correct creatures to match the instructor's symbols. The symbols may be found on the creatures, e.g., turtles, bellies in some embodiments. Some cases may vary the activity's difficulty level based on a patient's success by changing the settings, e.g., the number of creatures to choose from, introducing time requirements, extra visual stimuli, similar symbols, varying symbol colors, etc.

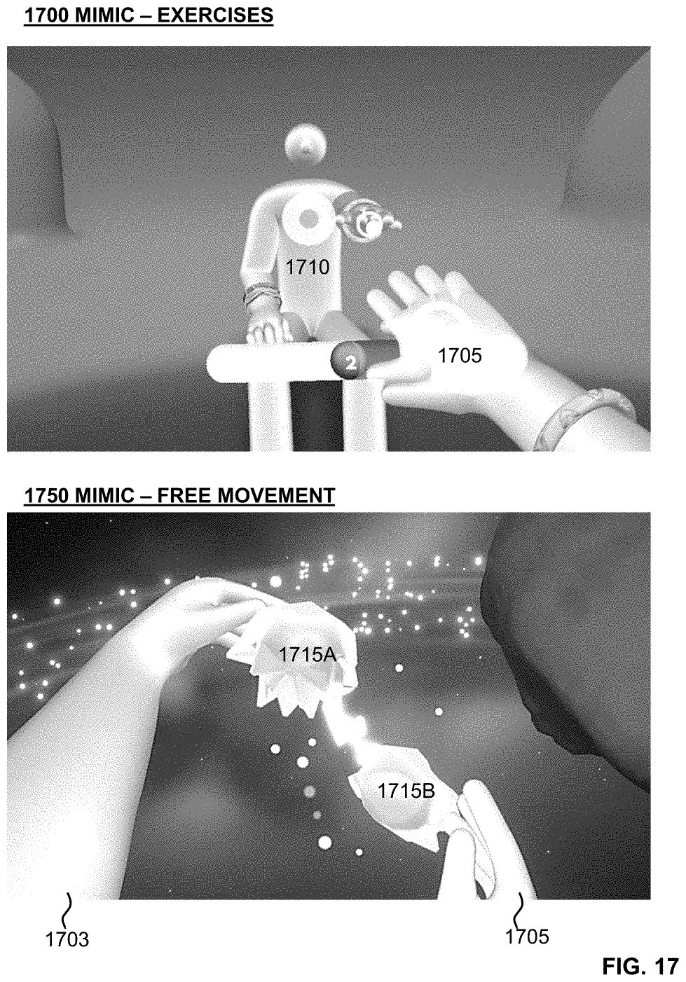

FIG. 17 depicts illustrative user interfaces for a VR therapy activity, Mimic, in accordance with some embodiments of the disclosure. Some embodiments may include the application or activity called Mimic. Mimic may help use joy, humor, and encouragement in order to lead patients through guided movement, focused distraction, and physics-based interaction. In some embodiments, therapists may guide their patients through exercises, or they may choose to have their patients follow a free movement experience. These exercises may be encouraged in some embodiments by auditory or visual indications of success, e.g., a character clapping for the user. They may also use signals to help indicate breaths for the patient as they go through the exercises. Some embodiments may use one of or both of left hand 1703 and right hand 1705.

Through the Mimic Exercises 1700 in some cases, patients may work on different areas, e.g., neck, upper back, shoulder, elbow and/or wrist. In some embodiments, there may be visual instructions from an NPC 1710 for a user to mimic, e.g., a silhouette of a hand where the patient is intended to raise their own. Some embodiments may encourage a patient as they go through multiple movements. Some embodiments may include sub-activities, such as having the user reach forward to toss items to knock down a wall.

In Free Movement 1750 exercises, simple movements may be encouraged, such as reaching to touch an object, to move it out of the way, or knock it apart. These activities may also include embodiments where a patient will put objects together in order to complete another task, e.g., pushing a shooting star into the distance. For instance, object 1715A and object 1715B may be combined and thrown into orbit as, e.g., a comet or meteor. Calming sounds may be used in some embodiments to aid in the calming nature of the activities. Some cases may also include rest moments to help patients take breaks in calm, low-stimulation environments.

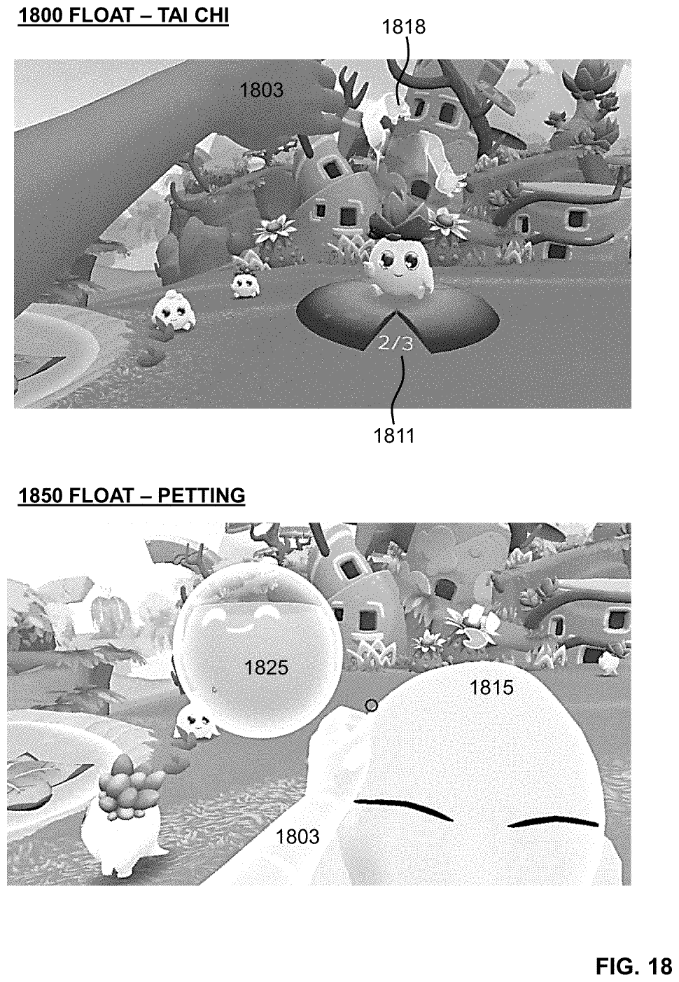

FIG. 18 depicts illustrative user interfaces for a VR therapy activity, Float, in accordance with some embodiments of the disclosure. In some embodiments, there may be an activity or activity called “Float” included. An embodiment may aim to build mental, emotional, and physical resilience through discovery, meditation and mindfulness. In some cases, patients may enjoy the calm of the activities and the power of choice that they have throughout the activities through their specific actions. In some embodiments, a patient may navigate to a task upon which they would like to focus, e.g., “Breathing,” “Tai Chi.”

Tai Chi 1800 is intended to help a patient relax and/or use muscle control for slow, smooth movements. Some embodiments may use instructions to direct their patients during use, for instance by having a script read to describe the task, having visualizations of the tasks (e.g., a silhouette of the movements of Tai Chi), or having creatures demonstrate the actions. In some instances of these activities, there may be encouragement for the users as they complete tasks e.g., characters cheering and jumping as the user completes a Tai Chi lesson or waving at the patient. The users may toss items at the characters using specific motions. Some embodiments may increase in difficulty as the patient progresses, such as requiring more precision in the activities or directing where to toss items. In some cases, users may earn badges as they progress to encourage their active engagement with the VR activity. The user may, in some embodiments interact directly with the creatures, e.g., petting them, and the creatures may respond to these interactions, e.g., making noises and emoting to portray enjoyment. For instance, in Petting 1850, an NPC 1815 may be petted by left hand 1903 and pet meter 1825 may fill up as the character's enjoyment rises. Float is designed to promote relaxation and comfortability in a virtual world.

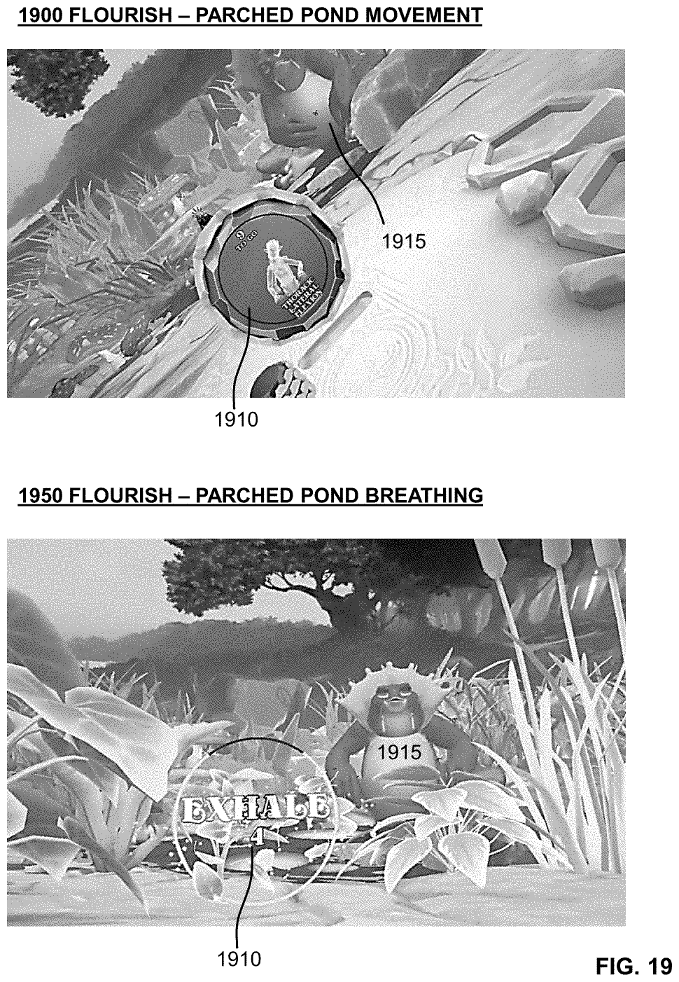

FIG. 19 depicts illustrative user interfaces for a VR therapy activity, Flourish, in accordance with some embodiments of the disclosure. Some embodiments may feature an activity named Flourish that may be a narrative-driven activity to help motivate patients to overcome resistance and actively engage in their recovery by using therapeutic motions that follow a story. Flourish may help patients with their range of motion and through increasing their abilities to complete ADL. Some embodiments may include activities: “Parched Pond,” “Floodfern Forrest,” and “Rootsoak Meadow.”

Some embodiments may require a patient to follow the storyline of the activity's character which may direct them to complete activities. In some embodiments, a non-playable character such as Vorn 1915, might vocally interact with a patient, e.g., talking to the patient or giving the patient instructions. In some embodiments, the instructions may take the form of auditory or visual queues, e.g., a silhouette for the user to mimic may appear on a rock 1910, or verbal instructions detailing what the user should be doing. In some cases, the user may obtain items within the VR world of the application to determine their success, and/or the user's successes or failures may be noted by visual or auditory queues, e.g., encouraging noises being played after an activity's completion, a bird entering the frame, or visually pleasing animations. Some cases may make the VR world more visually appealing and soothing as the patient completes tasks.

Parched Pond 1900 may encourage the user to complete specific movements in the physical world to complete the levels of the activity, e.g., Thoracic Lateral Flexion and completing breathing exercises describing how long to breath, hold, and exhales for. As the exercise progresses, a patient may move their progression in the activity forward or may compete with themselves to succeed. In some embodiments, such as Parched Pond 1950, a breathing exercise may be requested by meter 1910 as Vorn 1915 ask the patient to exhale for five seconds.

In Floodfern Forest, patients may work on their range of motion and functional reach. In some embodiments, tasks may include following instructions to create portions. Some embodiments of portions may reward a patient e.g., by making the forest more lush, growing special plants, helping creatures and plants grow healthier, etc. In some cases, a patient may be helping a character, e.g., Primordia, to help the environment flourish. Some embodiments may require a patient to create increasingly difficult portions, e.g., by requiring a patient to use their memory regarding the steps to create the portion, by expanding the motions required, by asking a patient to complete more complicated motions, reach further, or search for the correct ingredients.

In Rootsoak Meadow, some embodiments may encourage a patient to gain a character's trust by aiding them in their efforts to help the environment. Some embodiments may encourage a patient e.g., to cast spells of increasing difficulty, to remember certain actions in order to repeat them later or determine which spells to cast based on the current obstacle (e.g., to use a spell to light up a dark room or to locate an object).

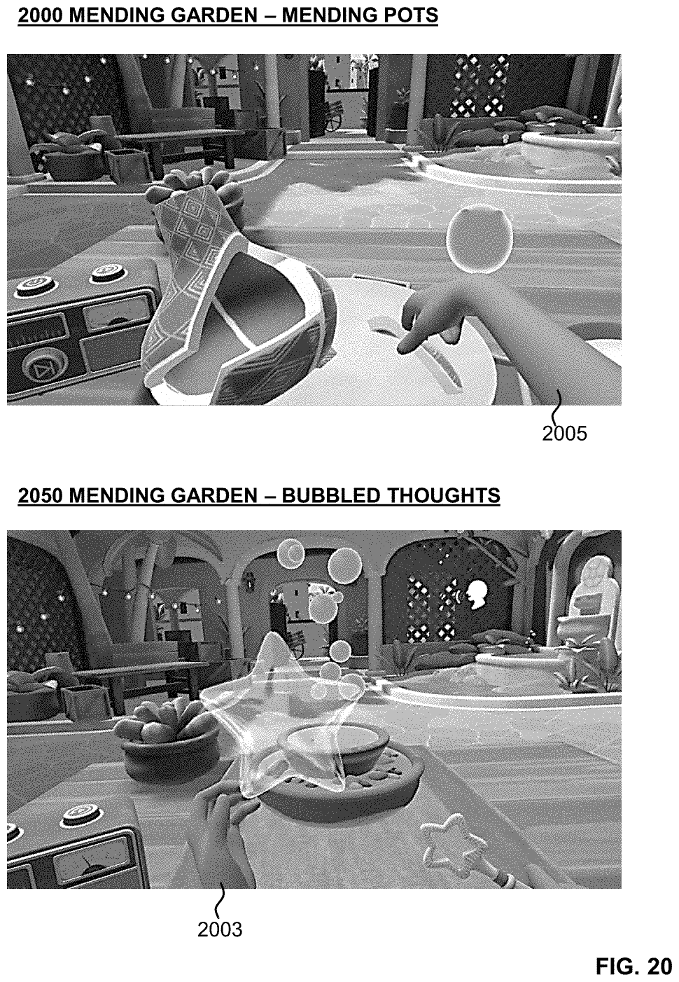

FIG. 20 depicts illustrative user interfaces for a VR therapy activity, Mending Garden, in accordance with some embodiments of the disclosure. Some embodiments may include an activity or activity called Mending Garden. Mending Garden may have the goal to help with mental disorders like depression or anxiety by invoking a patient's disposition and teaching depression therapy techniques. Some embodiments may attempt to create a calm and soothing environment for a patient to direct their thoughts to the activity and the encouragement of the tasks included in the activity. The embodiments may be designed to apply cognitive behavioral methodologies within a virtual space to encourage gentle physical engagement and meaningful reflection with a voice guide. In some cases, a therapist may be in control or supervising the use of the exercise, or the user may be participating the activity on their own (e.g., with virtual and/or lay supervision).

Some embodiments may include the exercise or activities “Mending Pots” and “Bubbled Thoughts” within the Mending Garden realm. Some cases may include a music capability that gives the user the opportunity to choose what background sounds there will be. A musical capability may let the user or therapist customize the experience further e.g., cycling through different songs to listen to, increasing or decreasing the volume, or returning to the main menu. Controls in the activity may also aid with functional reach capacities and precision of the user in order to choose what buttons they wish to push. The user may also have the capability to look around the world that they are in. Some cases may create virtual arms/hands for the user to use as controllers of the objects. Some embodiments may allow the users to choose options by looking at them and making a specific motion in order to control that choice, e.g., Mending Pots 2000 may include multiple resin choices which are outlines and upon the user's choice may guide them to place that choice in another position to activate the exercise. Guidance in some embodiments may be presented through the use of sockets and outlines of where objects are from or where they should go in order to be used. Some cases may include virtual buttons for multiple uses e.g., to determine the user's mood at the start or end of a session, to control sounds during a session, to choose an activity to play during the session. Some cases may include settings available within the activities through the use of an object in the environment, e.g., a journal menu where activities, calibration, and settings may be immediately available to the user.

In Mending Pots 2000, the user may be encouraged to put together broken pottery and reclaim its beauty by taking inspiration from the art of Kintsugi repair. This may have the goal to recognize common thought distortions from cognitive behavioral therapy and help a patient carve their own path. In repairing this pottery, the user may have multiple customizable features e.g., choosing what pottery they want to repair and what color they want to use to affix these repairs. Patients may also have multiple ways that they will be able to pick up the pieces of the broken pottery to repair it. In some cases, they might pick up a piece of pottery, decide that they picked it up incorrectly, and place it back on the surface where they originally grabbed it from in order to pick it up from a different position. The user may need to dip their pieces into the adhesive in the VR activity in order for the piece to connect to other pieces of the broken pottery. The pieces may require to be specifically placed together or come close to connecting before snapping back in place. In some embodiments, difficulty level can be dynamically increased by, e.g., requiring greater precision when the pieces connect, increasing the number of broken pieces, creating more irregular breaks in the pottery, having more complex patterns on the pottery, or creating goals for how many items need to be fixed. There may be audio and/or visual queues included in order to help the user determine their success in matching pieces.

In Bubbled Thoughts 2050, a patient may be encouraged to separate themselves from their thoughts by practicing thought diffusion and thought labelling. Some embodiments may also help patients with the activity inspired by Acceptance and Commitment therapy. The users may blow the bubble to help them visualize letting go of specific thoughts. The user may also have the ability to choose the shape of the bubbles that they blow.

Some embodiment may include a VR therapy activity called Virtual Athletic Club. Virtual Athletic Club may include activities and sub-activities like Paddle Pong, Bow Sling, and Power Punch. Each activity may require a patient to use proper form and, e.g., sub-optimal form results in a failure state, or regression of progress made. For instance, Paddle Pong may require a user to move his hand to mimic the form of a demonstrated virtual ping pong paddle. The exercises may have themes, including Jurassic Vaporwave, Cosmic Sea, and Digital Neon Safari. Virtual Athletic Club may have a journey mode where the patient practices form through a journey through the themes. Virtual Athletic Club may also have a mini-game mode to test the patient's skills and allow a patient to challenge for (personal) high scores. In some embodiments, patients can begin in journey mode as a warm-up, then apply their skills in smaller exercises. Patients may use the mini-game mode as a checkpoint of where they are, practice good form in journey mode, and then try activities again to see whether they have improved.

While the foregoing discussion describes exemplary embodiments of the present invention, one skilled in the art will recognize from such discussion, the accompanying drawings, and the claims, that various modifications can be made without departing from the spirit and scope of the invention. Therefore, the illustrations and examples herein should be construed in an illustrative, and not a restrictive sense. The scope and spirit of the invention should be measured solely by reference to the claims that follow.

1. A method of selecting a therapeutic virtual reality activity for a patient, the method comprising:
accessing a set of patient impairments;
accessing a plurality of activities, wherein one or more impairments of a plurality of impairments are associated with each of the plurality of activities;
comparing the set of patient impairments with the one or more impairments associated with each of the plurality of activities;
selecting an activity of the plurality of activities based on the comparison of the set of patient impairments with the one or more impairments associated with each of the plurality of activities; and
providing the selected activity of the plurality of activities.

2. The method of claim 1, wherein the accessing the set of patient impairments comprises:
accessing a patient profile associated with the patient;
accessing a plurality of impairments;
determining, based on the patient profile, the set of patient impairments from the plurality of impairments.

3. The method of claim 1 further comprising:
receiving input from the patient performing the selected activity;
accessing performance data; and
dynamically adjusting the selected activity based on the activity data and the input from the patient.

4. The method of claim 3, wherein dynamically adjusting the selected activity comprises dynamically adjusting at least one of the following for the selected activity: speed, difficulty, distractions, assistance, guidance, and instructions.

5. The method of claim 1, wherein determining the set of patient impairments from the plurality of impairments comprises:
accessing data describing each of the plurality of impairments;
matching data from the patient profile to the data describing each of the plurality of impairments; and
identifying a corresponding one of the plurality of impairments when data from the patient profile matches the data describing each of the plurality of impairments.

6. The method of claim 2, wherein the patient profile further comprises at least one of the following: sensor data, biometric data, and patient health history.

7. The method of claim 1, wherein each impairment of the set of patient impairments is associated with at least one of the following: a priority level and a weight.

8. The method of claim 1, wherein each of the one or more impairments associated with each of the plurality of activities is associated with at least one of the following: a priority level and a weight

9. The method of claim 1, wherein providing the selected activity of the plurality of activities further comprises providing a user interface comprising the selected activity.

10. The method of claim 1, wherein comparing the set of patient impairments with the one or more impairments associated with each of the plurality of activities further comprises counting matches between each of the set of patient impairments and each of the one or more impairments associated with each of the plurality of activities.

11. A system for selecting a therapeutic virtual reality activity for a patient, the system comprising:
input/output circuitry configured to:
access a set of patient impairments;
access a plurality of activities, wherein one or more impairments of a plurality of impairments are associated with each of the plurality of activities;
processing circuitry configured to:
compare the set of patient impairments with the one or more impairments associated with each of the plurality of activities;
select an activity of the plurality of activities based on the comparison of the set of patient impairments with the one or more impairments associated with each of the plurality of activities; and
provide the selected activity of the plurality of activities.

12. The system of claim 11, wherein the input/output circuitry is configured to access the set of patient impairments by:
accessing a patient profile associated with the patient;
accessing a plurality of impairments; and
the processing circuitry is further configured to determine, based on the patient profile, the set of patient impairments from the plurality of impairments.

13. The system of claim 11, wherein the input/output circuitry is further configured to:
receive input from the patient performing the selected activity;
access performance data; and
processing circuitry is further configured to dynamically adjust the selected activity based on the activity data and the input from the patient.

14. The system of claim 13, wherein the processing circuitry is further configured to dynamically adjust the selected activity by dynamically adjusting at least one of the following for the selected activity: speed, difficulty, distractions, assistance, guidance, and instructions.

15. The system of claim 11, wherein the processing circuitry is further configured to determine the set of patient impairments from the plurality of impairments by:
accessing data describing each of the plurality of impairments;
matching data from the patient profile to the data describing each of the plurality of impairments; and
identifying a corresponding one of the plurality of impairments when data from the patient profile matches the data describing each of the plurality of impairments.

16. The system of claim 12, wherein the patient profile further comprises at least one of the following: sensor data, biometric data, and patient health history.

17. The system of claim 11, wherein each impairment of the set of patient impairments is associated with at least one of the following: a priority level and a weight.

18. The system of claim 11, wherein each of the one or more impairments associated with each of the plurality of activities is associated with at least one of the following: a priority level and a weight

19. The system of claim 11, wherein the processing circuitry is further configured to provide the selected activity of the plurality of activities by providing a user interface comprising the selected activity.

20. The system of claim 11, wherein the processing circuitry is further configured to compare the set of patient impairments with the one or more impairments associated with each of the plurality of activities further by counting matches between each of the set of patient impairments and each of the one or more impairments associated with each of the plurality of activities.

21.-60. (canceled)
